SEQUENCE LISTING (4 sequences)

SEQ ID NO: 1 - DNA, 20 bp, synthetic construct
  source 1..20: mol_type = other DNA
tcggagtggtgcgtcaaaga

SEQ ID NO: 2 - DNA, 20 bp, synthetic construct
  source 1..20: mol_type = other DNA
cccggctgcccccccttgcg

SEQ ID NO: 3 - skipped

SEQ ID NO: 4 - DNA, 30 bp, synthetic construct
  source 1..30: mol_type = other DNA
cggggccttagcgccggggccttagtgggc

(19) United States
(12) Patent Application Publication
(10) Pub. No.: US 2024/0266000 A1
(43) Pub. Date: Aug. 8, 2024

(54) DNA ENCODING AND DECODING METHOD USING DEGENERATE NUCLEOTIDE

(71) Applicant: Keun Woo LEE, Yongin-si (KR)

(72) Inventors: Keun Woo LEE, Yongin-si (KR); Seong Jun SEO, Suwon-si (KR); Su Youn PARK, Suwon-si (KR)

(73) Assignee: Keun Woo LEE, Yongin-si (KR)

(21) Appl. No.: 18/433,401

(22) Filed: Feb. 6, 2024

(30) Foreign Application Priority Data
Feb. 7, 2023 (KR) 10-2023-0016238
Feb. 15, 2023 (KR) 10- 2023-0019722
Feb. 20, 2023 (KR) 10-2023- 0022317
Feb. 23, 2023 (KR) 10-2023- 0023971

(51) Int. Cl. G16B 30/20

(52) U.S. Cl. CPC G16B 30/20

(57) ABSTRACT

The present specification discloses a DNA encoding and decoding method capable of converting and storing data more efficiently, wherein the DNA encoding method according to the present specification may convert binary data into sequence information using basic base values and degenerate base values, the degenerate base value may be a combination of at least two basic base values among four basic bases and the DNA decoding method according to the present specification may divide the basic base value and the degenerate base value into binary data according to base types for the same position in a plurality of DNA strands.

### CROSS REFERENCE TO RELATED APPLICATIONS

This application claims the priority benefit of Korean Patent Application No. 10-2023-0016238 filed on Feb. 7, 2023, Korean Patent Application No. 10-2023-0019722 filed on Feb. 15, 2023, Korean Patent Application No. 10-2023-0022317 filed on Feb. 20, 2023 and Korean Patent Application No. 10-2023-0023971 filed on Feb. 23, 2023, each of which is hereby incorporated by reference herein in its entirety.

### SEQUENCE LISTING

This application contains a Sequence Listing submitted via USPTO Patent Center and hereby incorporated by reference in its entirety. The Sequence Listing is named SEQCRF_4120-001.xml, created on Jan. 24, 2024, and 3,787 bytes in size.

### BACKGROUND OF THE INVENTION

The present invention relates to DNA molecule-based information storage technology, and more particularly, to encoding and decoding between binary data and base sequences.

The contents described in this section merely provide background information about the embodiments described herein, but do not necessarily constitute the conventional technology.

For magnetic tapes widely used as long-term recording media, the data storage lifespan is limited to about 10 years, so that maintenance and management costs are continuously required. In the case of semiconductor storage devices, HDD and SSD are representative. The HDD lifespan is mainly 5 years, and when the number of data access frequencies is used less than once per quarter, they have a lifespan of about 10 years, but they are very vulnerable to impact and the maximum capacity value is limited. In the case of SSD, the lifespan is relatively shorter than HDD, although it is strong to impact. Recently, the explosive amount of data is overloaded by exceeding the capacity of the storage medium, and it reaches the limit of the data storage density of the existing information storage medium, so that a new type of storage device has been needed.

Among attempts to develop a new storage medium, attempts to develop a new storage medium using DNA have been made. When DNA is used as a storage medium, the data storage density, which is a disadvantage of the existing storage medium, can be excellently exceeded, and information can be stably stored for a long time even with physical impact.

As is well known, DNA is contained in a cell that is the smallest unit of a living body, and contains all genetic information. All living organisms grow and move as if they are programmed according to the information contained in DNA. In the case of humans, DNA contained in one single cell is composed of 30 billion pairs of base sequences, and the capacity is about 1 TB if the size of the genetic information that has been completely decoded is converted. In addition, two strands of DNA having a width of 2 nm and a length of 3 m are contained in one single cell. Therefore, DNA, which is the next-generation biostorage that can be stored in theory of EB (10\hat{ }18) or more, is very suitable as a biomaterial for very intensively storing information. In addition, the storage lifespan is 1,000 years or more, and it is expected that it can be stored at a low cost.

FIG. 1 is a conceptual diagram of information storage using a DNA base sequence.

Referring to FIG. 1, binary data to be stored is encoded by nucleotides A (adenine), T (thymine), G (guanine), and C (cytosine). The DNA is synthesized according to the encoded base sequence, and the synthesized DNA molecule is stored. Then, the stored DNA molecule is selected through a search (Retrieval), the selected DNA molecule is analyzed (Sequencing), and the binary data is decoded according to the analyzed base sequence.

DNA sequencing technology is expected to have a great effect in realizing disease treatment, particularly personalized medicine. Currently, non-nanopore technology has become commercialized as a large sample preparation and complicated algorithms for data processing, and a short read length. As a result, DNA sequence technology has recently been grafted to the temporary single-molecule nanopore technology since the development of the 3rd generation nucleotide sequence technology.

Nanopores refer to pores having a nanometer (nm, 10-9 m) size, and from 1996 to the first nanopore paper published in PNAS (Proceedings of the National Academy of Sciences of the United States of America), nanopore-based detection for single molecules has been emerged as one of the DNA nucleotide sequence technologies. An important advantage of such nanopore-based technology is that chemical labeling is not necessary, and it is possible to read long-length DNA and to have excellent information throughput.

Nanopore-based technology uses the principle that particles having a slightly smaller size than the pore pass through the pore due to the applied external voltage. The nanometer-sized pore is embedded in a biological membrane or formed in a solid film such as graphene, and thus a reservoir containing a conductive electrolyte is separated into a cis-compartment and a trans-compartment. When a voltage is applied, the ions of the electrolyte solution pass through the pores in the principle of electrophoresis and generate an ionic current signal. When the pores are blocked by an analyte such as anionic DNA molecules formed in the cis-compartment, the current passing through the nanopores is blocked and the current signal is disturbed. When the amplitude of the current generated in the series of processes and the reaction duration are statistically analyzed, the physical and chemical characteristics of the target molecule can be grasped.

Biological nanopores were used as a material of a membrane forming a nanopore. As biological nanopores, Oxford Nanopore technology is typically known. The Oxford Nanopore technology applies a method of analyzing the potential change according to each nucleotide sequence generated as a single strand of DNA passes through the nanosized pores. A complex where DNA and enzyme are bound is attached to a motor protein on a thin membrane, and exonuclease is bound to the end of DNA of a double-stranded structure to produce single-stranded DNA, and the single-stranded DNA thus produced passes through the pores of a membrane made of a protein and generates an electrical signal, which is measured using a chip. Such biological nanopores can generate accurate electrical signal with high sensitivity in DNA nucleotide sequence technology and provide excellent reproducibility.

### SUMMARY OF THE INVENTION

The present invention has been made in an effort to provide a separator for a redox flow battery which enables ions to easily move in an electrolytic solution by providing an ionomer coating layer with a diameter of ion clusters adjusted on at least one surface of a porous substrate, a manufacturing method therefor, and a redox flow battery comprising the same.

The present specification aims to provide a DNA encoding and decoding method capable of converting and storing data more efficiently.

In addition, the present invention aims to provide a single-stranded DNA structure analysis apparatus and a single-stranded DNA structure analysis method using the same, for analyzing single-stranded DNA structure where data is encoded and analyzing encoded data.

In biological nanopore technology, in the case of protein nanopores, it is not easy to manipulate the size and charge of the pores according to the intention of the researcher, and even a small change in temperature or the pH of the hydrogen ion concentration (the potential hydrogene) when the protein is used, has a great influence on the compliance and biological activity of the biological nanopores, so it is sensitive to external environmental conditions.

Therefore, the present invention can be proposed to solve the problems that may occur in the DNA chip where the nanopore technology is grafted, including the above-described problems.

The present invention is a task to be solved, and it is to provide a DNA chip where a semiconductor technology is grafted to effectively adjust the diameter of the nanopores and a manufacturing method thereof.

In addition, the present invention is another task to be solved, and it is to provide a data storage and reading method for the DNA chip where the semiconductor technology is grafted, which is newly proposed in the present specification.

In addition, the present invention is another task to be solved, and it is to provide an expandable DNA chip capable of increasing the data storage capacity of the DNA chip proposed in the present specification.

The present specification is not limited to the above-mentioned tasks, and other tasks not mentioned will be clearly understood by those skilled in the art from the following description.

The DNA encoding method according to the present specification to solve the above problem may include (a) converting, by the processor, binary data into sequence information using a basic base value and a degenerate base value.

According to an embodiment of the present specification, the degenerate base value may be a combination of at least two basic base values among four basic bases.

The DNA encoding method according to the present specification may further include (b) generating, by the processor, a kind of DNA strand to be generated in the DNA synthesis step according to the number of basic base values used to express the degenerate base value.

The DNA encoding method according to the present specification may further include (c) distributing, by the processor, the total number of DNA strands to be generated in the DNA synthesis step uniformly according to the kind of DNA strands to be generated in the DNA synthesis step.

The DNA encoding method according to the present specification may be implemented in the form of a computer program written in a computer to perform each step of the DNA encoding method and recorded in a computer-readable recording medium.

The DNA encoding apparatus according to the present specification to solve the above problem may include a processor that converts binary data into sequence information using a basic base value and a degenerate base value.

According to an embodiment of the present specification, the degenerate base value may be a combination of at least two base values among four basic bases.

According to an embodiment of the present specification, the processor may further generate, by the processor, a kind of DNA strand to be generated in the DNA synthesis step according to the number of basic base values used to express the degenerate base value.

According to an embodiment of the present specification, the processor may further distribute, by the processor, the total number of DNA strands to be generated in the DNA synthesis step uniformly according to the kind of DNA strands to be generated in the DNA synthesis step.

The DNA encoding apparatus according to the present specification may be an element of a DNA information storage system that includes a DNA encoding apparatus and a DNA synthesis apparatus that synthesizes DNA according to the base sequence information output from the DNA encoding apparatus.

The DNA decoding method according to the present specification to solve the above problem may include (a) receiving, by the processor, base sequence analysis information for a plurality of DNA strands; (b) dividing, by the processor, a basic base value and a degenerate base value according to the kind of bases for the same position in a plurality of DNA strands; and (c) converting, by the processor, a basic base value and a degenerate base value into binary data.

According to an embodiment of the present specification, the degenerate base value may be a combination of at least two base values among four basic bases.

According to an embodiment of the present specification, the step (b) may be divided into a basic base value and a degenerate base value according to the ratio of bases for the same position in a plurality of DNA strands.

According to an embodiment of the present specification, the step (b) may be excluded from the base value and the degenerate base value a a less than the preset reference number.

The DNA decoding method according to the present specification may be implemented in the form of a computer program written to perform each steps of the DNA decoding method.

In order to solve the aforementioned problem, the DNA decoding apparatus according to the present specification may include a processor that receives base sequence analysis information for a plurality of DNA strands, divides the DNA strands into basic base values and degenerate base values according to base types for the same position, and converts the basic base values and the degenerate base values into binary data.

According to an embodiment of the present specification, the degenerate base value may be a combination of at least two basic base values among four basic bases.

According to an embodiment of the present specification, the processor may be divided into basic base values and degenerate base values according to the ratio of base types for the same position in the plurality of DNA strands.

According to an embodiment of the present specification, the processor may exclude less than a preset reference number of bases from the basic base values and the degenerate base values.

The DNA decoding apparatus according to the present specification may be an element of a DNA information storage system that includes a DNA analysis apparatus that analyzes a base sequence of a DNA molecule and outputs base sequence information; and a DNA decoding apparatus.

Another DNA encoding method according to the present specification to solve the aforementioned problem may include (a) storing a data transformation matching table that converts data into base values by a processor; (b) first converting binary data into any one of four binary data, eight binary data, and hexadecimal data by the processor; and (c) second converting the first converted data into sequence information using basic base values and degenerate base values according to the data transformation matching table by the processor.

According to an embodiment of the present specification, the degenerate base value may be a combination of at least two basic base values among four basic bases.

The DNA encoding method according to the present specification may further include (d) generating a type of DNA strands to be generated in the DNA synthesis step according to the number of basic base values used to express the degenerate base values.

The DNA encoding method according to the present specification may further include (e) uniformly distributing the total number of DNA strands to be generated in the DNA synthesis step according to the type of DNA strands to be generated in the DNA synthesis step.

According to an embodiment of the present specification, the data transformation matching table may include transformation base values that match each data according to a preceding base value.

According to an embodiment of the present specification, the preceding base value and the transformation base value of the data transformation matching table may be a combination of two base values including at least any one of basic base values and degenerate base values.

According to an embodiment of the present specification, the transformation base value of the data transformation matching table may have a first base value different from the second base value among the preceding base values.

The DNA encoding method according to the present specification may be implemented in the form of a computer program written on a computer readable recording medium by writing the computer to perform each steps of the DNA encoding method.

The DNA encoding apparatus according to the present specification for solving the above-described tasks may include a storage unit that stores a data transformation matching table that converts data into base values; and a processor that first-converts binary data into any one of four binary, eight binary, and sixteen binary data, and second-converts the first-converted data into sequence information using basic base values and degenerate base values.

According to an embodiment of the present specification, the degenerate base value may be a combination of at least two or more basic base values among four basic bases.

According to an embodiment of the present specification, the processor may further generate the type of DNA strand to be generated in the DNA synthesis step according to the number of basic base values used to express the degenerate base value.

According to an embodiment of the present specification, the processor may further distribute the total number of DNA generation strands to be generated in the DNA synthesis step uniformly according to the type of DNA strands to be generated in the DNA synthesis step.

According to an embodiment of the present specification, the data transformation matching table may consist of transformation base values that match each data according to a prior base value.

According to an embodiment of the present specification, the prior base value and the transform base value of the data transformation matching table may be a combination of two base values including at least any one of a basic base value and a degenerate base value.

According to an embodiment of the present specification, the transformation base value of the data transformation matching table may have a first base value different from the second base value among the preceding base values.

The DNA encoding apparatus according to the present specification may be one component of a DNA information storage system including a DNA encoding apparatus and a DNA synthesizing apparatus for synthesizing DNA according to the base sequence information output from the DNA encoding apparatus.

Another embodiment of the present invention provides a single-stranded DNA structure analysis apparatus comprising: a DNA data storage unit storing a single-stranded DNA structure; a DNA data receiving unit receiving the analyzed single-stranded DNA structure; a separation plate including a nanochannel communicating with the DNA data storage unit; and a first capacitor electrode and a second capacitor electrode provided to be spaced apart by the nanochannel and electrically connected via a capacitance sensing unit.

Another embodiment of the present invention provides a single-stranded DNA structure analysis method using the single-stranded DNA structure analysis apparatus, comprising: (a) receiving an electrolyte solution in the DNA data storage unit and the DNA data receiver of the single-stranded DNA structure analysis apparatus; (b) moving the single-stranded DNA structure of the DNA data storage unit to the DNA data receiver through the nanochannel; and (c) analyzing the base sequence of the single-stranded DNA structure passing through the nanochannel using the base monitoring current value Im of the single-stranded DNA structure according to Equation 1 below.

$${I}_{m} = \frac{{C}_{b}}{{C}_{s}} \cdot \frac{d V}{d t} \qquad equation 1$$

In Equation 1, Cs is the capacitance value in a state in which the nanochannel is filled with the electrolyte solution, Cb is the capacitance value according to the intrinsic permittivity of each base of the single-stranded DNA structure passing through the nanochannel, and V is the voltage.

According to another embodiment of the present invention, the present invention provides a semiconductor-based DNA chip comprising: first and second fluid compartments filled with electrolyte; and silicon-based nanopore membranes formed between the first fluid compartment and the second fluid compartment, and the first and second fluid reservoirs communicate with each other.

In addition, the first fluid partitioning portion may include a drain electrode formed in a lower portion of the first fluid reservoir, and the second fluid partitioning portion may include a control electrode and a source electrode formed on an upper portion of the nanopore membrane to surround the second fluid reservoir.

In addition, the first fluid partitioning portion may include a lower insulating film formed to cover the lower portion of the nanopore film and surround the first fluid reservoir, and the second fluid partitioning portion may include an upper insulating film formed in the lower portion of the control electrode to cover the upper portion of the nanopore film and surround the lower portion and the bottom portion of the second fluid reservoir.

In addition, the nanopore film may be formed of a silicon nitride film.

In addition, the single nanopore may be formed in a diameter of 1 to 5 nm.

In addition, one or a plurality of control electrodes may be stacked.

In addition, according to another embodiment of the present invention, the present invention provides a method of manufacturing a semiconductor-based DNA chip, comprising: forming a lower electrode layer, a sacrificial insulating layer, a lower insulating layer, a silicon-based nanopore film, an upper insulating layer, a first upper electrode layer, a separation insulating layer, and a second upper electrode layer on a base substrate; forming a hole by etching the second upper electrode layer, the separation insulating layer, and the first upper electrode layer; forming a spacer on an inner wall of the hole; forming a single nanopore by etching the upper insulating layer, the nanopore film, and the lower insulating layer exposed between the spacers by using the spacers; removing the spacers; and selectively removing the sacrificial insulating layer partitioned by the lower insulating layer and the lower electrode layer.

In addition, the process of selectively removing the sacrificial insulating layer may be performed by introducing an etching solution into the sacrificial insulating layer while embedding the single nanopore and the hole using the sacrificial insulating layer.

In addition, the sacrificial insulating film may embed the hole using a metal material after forming a spacer made of a silicon oxide film on the inner wall of the hole.

In addition, the sacrificial insulating film may embed a part of the hole using a silicon nitride film, and then the remainder may embed the hole using a silicon oxide film.

In addition, the nanopore film may be formed of a silicon nitride film.

In addition, according to another embodiment of the present invention, in the data storage method of the semiconductor substrate DNA chip, the method includes applying a positive voltage to the control electrode to attach and fix the single stranded DNA present in the second fluid reservoir to the control electrode.

In accordance with another aspect of the present invention, there is provided a method of reading data from a semiconductor substrate DNA chip, the method comprising: applying a negative voltage to a control electrode to detach a single strand DNA attached to the control electrode; applying a positive voltage to the drain electrode, and applying a ground voltage to the source electrode to induce a flow of electric charge in an electrolyte solution filled in a first and second fluid reservoirs; and sensing a flow of electric current attributable to the flow of electric charge in the electrolyte solution to sense a change in amount of electric current that changes when the single strand DNA detached from the control electrode passes through a single nanopore.

In accordance with another aspect of the present invention, there is provided a method of storing and reading data from a semiconductor substrate DNA chip, the method comprising: applying a positive voltage to a control electrode to detach a single strand DNA present in a second fluid reservoir to the control electrode; applying a negative voltage to the control electrode to detach a single strand DNA attached to the control electrode; applying a positive voltage to the drain electrode, and applying a ground voltage to the source electrode to induce a flow of electric charge in an electrolyte solution filled in a first and second fluid reservoirs; and sensing a flow of electric current attributable to the flow of electric charge in the electrolyte solution to sense a change in amount of electric current that changes when the single strand dan detached from the control electrode passes through a single nanopore.

Other details of the present invention are included in the detailed description and drawings.

According to the present specification, it is possible to provide a DNA encoding and decoding method capable of converting and storing data more efficiently.

In addition, when the single-stranded DNA structure analysis method according to one embodiment of the present invention is used, the base of the single-stranded DNA structure can be accurately analyzed, and thus the loss of data encoded in the single-stranded DNA structure can be minimized and decoded.

In addition, when the single-stranded DNA structure analysis method according to one embodiment of the present invention is used, the analysis of the information encoded in the single-stranded DNA structure can be more accurately performed through a first analysis using the current sensing unit and a second analysis using the capacitance sensing unit.

According to the DNA chip according to the present invention, by configuring a single nanopore using a silicon-based nanopore film, the diameter of the single nanopore can be easily fine-adjusted by grafting semiconductor technology compared to conventional biological nanopores.

In addition, according to the DNA chip according to the present invention, by forming a nanopore film on a silicon-based material that can be applied to the semiconductor technology, the thickness of the nanopore film can be more precisely controlled.

In addition, according to the DNA chip according to the present invention, by forming a nanopore film on a silicon-based material that can be applied to the semiconductor technology, the manufacturing is simple compared to conventional biological nanopores, and the number of control electrodes can be easily expanded using a deposition and etching (patterning) process, whereby addressable and the data storage capacity can be increased.

In addition, according to the DNA chip according to the present invention, by forming electrode layers in the first and second fluid compartments formed vertically around the nanopore film, bias voltage can be applied to simultaneously implement the data storage and reading of single-stranded DNA.

In addition, according to the DNA chip according to the present invention, it can be used as a recording device that records data using single-stranded DNA. In particular, it can be retained for a long time without modification.

The effects of the present invention are not limited to the above-mentioned effects, and other effects that are not mentioned can be clearly understood by those skilled in the art from the following description.

### BRIEF DESCRIPTION OF DRAWINGS

FIG. 1 is a conceptual diagram of information storage using a DNA base sequence.

FIG. 2 is a reference diagram of a DNA information storage system according to the present specification.

FIG. 3 is a flowchart of a DNA encoding method according to the present specification.

FIG. 4 is an example of encoding using only a conventional base.

FIG. 5 is an example of encoding according to an embodiment of the present specification.

FIG. 6 is an example of a DNA strand generated when a D2 degenerate base is used.

FIG. 7 is an example of 10 DNA strands distributed according to the type of DNA strands.

FIG. 8 is a flowchart of a DNA decoding method according to the present specification.

FIG. 9 is a flowchart of a DNA encoding method according to another embodiment of the present specification.

FIG. 10 is a table showing whether or not overlaps within two base value combinations.

FIG. 11 is an example of a data transformation matching table according to an embodiment of the present specification.

FIGS. 12 to 15 are examples of a secondary transformation.

FIG. 16 is a schematic diagram of a single-stranded DNA structure analysis device according to an embodiment of the present invention.

FIG. 17 is a cross-sectional view of a semiconductor-based DNA chip according to an embodiment of the present invention.

FIG. 18 is a diagram showing driving characteristics of a semiconductor substrate DNA chip according to an embodiment of the present invention.

FIG. 19 is a conceptual diagram of a data reading operation of a DNA chip according to an embodiment of the present invention.

FIG. 20 is a cross-sectional view of a semiconductor-based DNA chip according to another embodiment of the present invention.

FIGS. 21 to 44 are processes showing a semiconductor-based DNA chip manufacturing method according to an embodiment of the present invention.

FIG. 45 is a plan view of a DNA chip manufactured through FIGS. 21 to 44.

FIGS. 46 and 47 are cross-sectional perspective views of the DNA chip shown in FIG. 45.

### DETAILED DESCRIPTION OF THE INVENTION

The advantages and features of the invention disclosed in the specification, and the methods of achieving them will become apparent by reference to the embodiments described in detail below together with the accompanying drawings. However, the present specification is not limited to the embodiments disclosed below, but may be implemented in various forms different from each other, and the present embodiments are provided so that the disclosure of the present specification is thorough, and the scope of the present specification is fully known to those skilled in the art to which the present specification belongs (hereinafter, ‘those skilled in the art’), and the scope of the present specification is defined only by the scope of the claims.

The terminology used in the specification is for the purpose of describing the embodiments and is not intended to limit the scope of the present specification. In the present specification, the singular forms include plural forms unless specifically mentioned in the phrase. As used in the specification, “comprises” and/or “comprising” does not exclude the presence or addition of one or more other components other than the mentioned components.

Throughout the specification, the same reference numerals refer to the same components, and “and/or” includes each and all combinations of one or more of the mentioned components. Although “first”, “second”, and the like are used to describe various components, these components are not limited by these terms. These terms are used to distinguish one component from another. Therefore, it is also understood that the first component mentioned below may be a second component within the technical spirit of the present invention.

Unless otherwise defined, all terms (including technical and scientific terms) used in the specification may be used as meanings that can be commonly understood by those skilled in the art to which the present specification belongs. In addition, the terms defined in commonly used dictionaries are not ideally or excessively interpreted unless specifically defined.

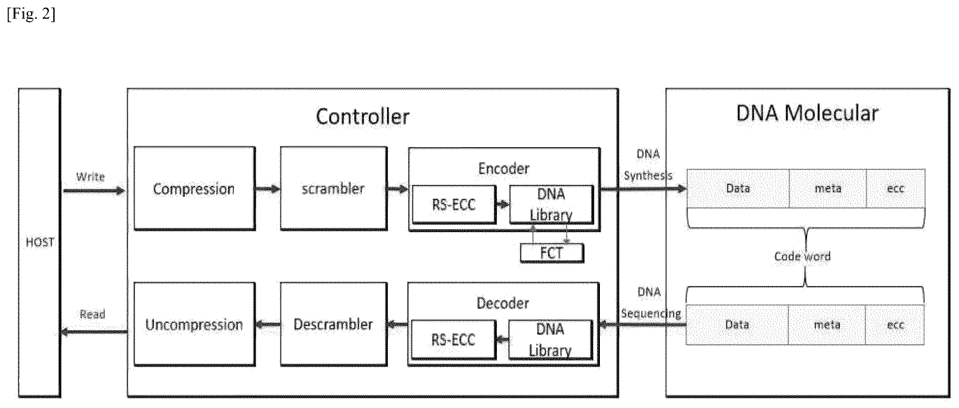

FIG. 2 is a reference diagram for a DNA information storage system according to the present specification.

Referring to FIG. 2, the DNA information storage system is largely divided into a controller and a DNA molecular unit. When the controller receives a request (Write) for storing information from the host, the controller may compress (Binary) data and mix the compressed data (Scrambler). The reason for mixing data is to prevent the same nucleotide sequence from being repeated when binary data is subsequently replaced into the nucleotide sequence. The data completed in the mixing process may be added an error correction code (ECC). Then, the binary data may be converted into nucleotide sequence information (DNA Library). The DNA molecular unit may synthesize the actual DNA molecule according to the data converted into the nucleotide sequence.

Then, when the request for read information stored as the DNA molecule, the DNA molecular unit may analyzes the nucleotide sequence. The controller converts the binary data into the binary data according to the analyzed nucleotide sequence and corrects the data errors using the error correction code (ECC) (Encoder). The binary data corrected by errors can be provided by solving the mixed state (Descrambler), releasing compression (Decompression), and providing the original binary data (information).

On the other hand, biologically, a DNA double helix structure in a cell is formed, but a DNA molecule synthesized according to the present specification, that is, a DNA strand, may be a single DNA strand. However, it is not excluded that the DNA strand according to the present specification has a double helical structure.

In addition, the DNA information storage system according to the present specification can synthesize a plurality of DNA strands to store one data. In other words, a plurality of DNA strands corresponding to one binary data may be used. This may be to reduce the possibility of errors in stable information storage, repeated information retrieval, and analysis.

Hereinafter, terms for describing the DNA encoding and decoding method according to the present specification are described in advance. In this specification, “binary data” means data consisting of 1 and 0.

As used herein, the term “basic base” refers to four nucleotides A (adenine), T (thymine), G (guanine), and C (cytosine).

In this specification, “basic base value” refers to data corresponding to basic bases in the process of information processing rather than actual nucleotides.

As used herein, “degenerate base” refers to base information expressed by a combination of four basic bases. For example, a total of six information that can be expressed by a combination of two bases among the four basic bases is 6. In addition, a total of four information that can be expressed by three base combinations among the four basic bases is four. In addition, a total of one information that can be expressed by a combination of four bases among the four basic bases is 1. In this specification, the degenerate base expressed by two base combinations among four basic bases is denoted as “D1”, the degenerate base expressed by three base combinations among four basic bases is denoted as “D2”, and the degenerate base expressed by four base combinations among four basic bases is denoted as “D3”.

As used herein, the term “degenerate base value” refers to data corresponding to a combination of basic bases in the process of information processing rather than actual nucleotides.

Each notation of the basic base and the degenerate base is shown in Table 1 below.

TABLE 1

| Title | | Notation | Constituent |
|---|---|---|---|
| Standard base | | A | |
| | | T | |
| | | C | |
| | | G | |
| Degenerate base | D₁ | W | A, T |
| | | S | G, C |
| | | R | A, G |
| | | Y | T, C |
| | | M | A, C |
| | | K | T, G |
| | D₂ | B | T, G, C |
| | | V | A, G, C |
| | | H | A, T, C |
| | | D | A, T, G |
| | D₃ | N | A, T, G, C |

In this specification, the total number of DNA generated strands means the number of DNA strands generated to store one binary data. For example, the DNA strands may be 2, 10, 50, 100 and so on to store one binary data. The total number of DNA strands to be generated in the DNA synthesis step can be set in advance according to the storage environment, policy, etc.

In this specification, the type of the DNA strand means the number of DNA strands having different sequence information among the DNA strands generated to store one binary data. For example, we will assume generating 10 DNA strands to store binary data called ‘00011011’. In order to store binary data ‘00011011’, if 5 strands of ‘AGCT’ DNA and 5 strands of ‘TCGA’ DNA are generated, the type of DNA is two. And, on the other hand, in this specification, the order of bases determines the direction of reading from 5′ to 3′ in the forward direction.

Meanwhile, the DNA encoding method according to the present specification may be implemented in the form of a computer program recorded on a computer-readable recording medium prepared and recorded in a computer to perform each steps to be described below. When the DNA encoding method according to the present specification is implemented in the form of a computer program, each step may be executed by a processor.

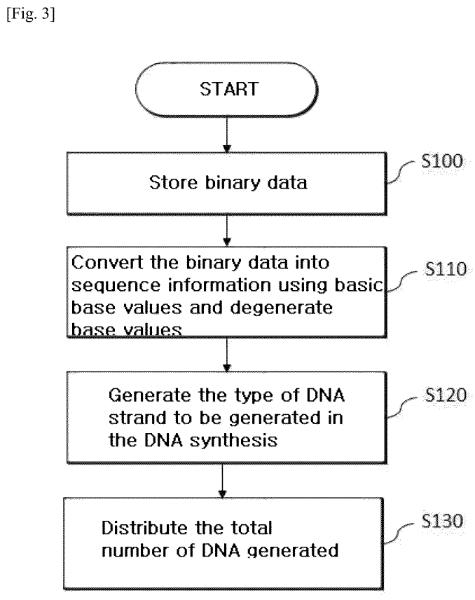

FIG. 3 is a flowchart of a DNA encoding method according to the present specification.

Referring to FIG. 3, first, in step s100, the processor may store binary data. The binary data can be the same as the original data, can be compressed data of the original data, or can be encrypted data. In addition, the binary data may be a payload of a file packet, or may be data added with an error correction code (ECC) or tag.

In the next step s110, the processor may convert the binary data into sequence information using basic base values and degenerate base values. As described above, the degenerate base value may be a combination of at least two or more basic base values among four basic bases. To help understand, an example of converting binary data using the four basic bases in Table 1 (A, T, C, G) and W, S, R, and Y of the degenerate bases will be described. Since a total of eight base values are used, it is assumed that each base value corresponds to binary data of 3 bits, and each base value corresponds to the values of Table 2 below.

TABLE 2

| Notation | Constituent | binary |
|---|---|---|
| A | | 000 |
| T | | 001 |
| C | | 010 |
| G | | 011 |
| W | A, T | 100 |
| S | G, C | 101 |
| R | A, G | 110 |
| Y | T, C | 111 |

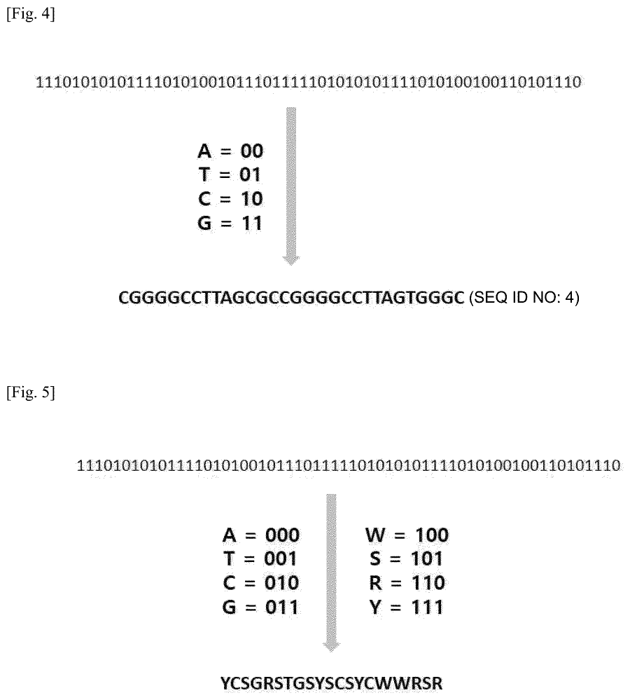

FIG. 4 is an example of encoding only with conventional bases.

Referring to FIG. 4, examples of converting the example binary data “111010101011110101001011101111101010101111010100100110101110” by only four basic bases are shown. It can be seen that the existing 60-bit data has been converted into 30 bases.

FIG. 5 is an example of encoding according to an embodiment of the present specification.

Referring to FIG. 5, the same binary data shown in FIG. 4 is converted into basic base values and degenerate base values according to Table 2. It can be seen that the same 60-bit data has been converted into 20 base values. It is possible to convert the same binary data more efficiently.

In the next step s120, the processor may generate the type of DNA strand to be generated in the DNA synthesis step according to the number of basic base values used to express the degenerate base value. When a degenerate base (D1) expressed as a combination of two bases out of four basic bases is used, at least two kinds of DNA strands can be produced. When degenerate base (D2) expressed as a combination of three bases out of four basic bases is used, at least three kinds of DNA strands can be produced. When a degenerate base (D3) expressed as a combination of four bases among the four bases is used, at least four kinds of DNA strands can be produced.

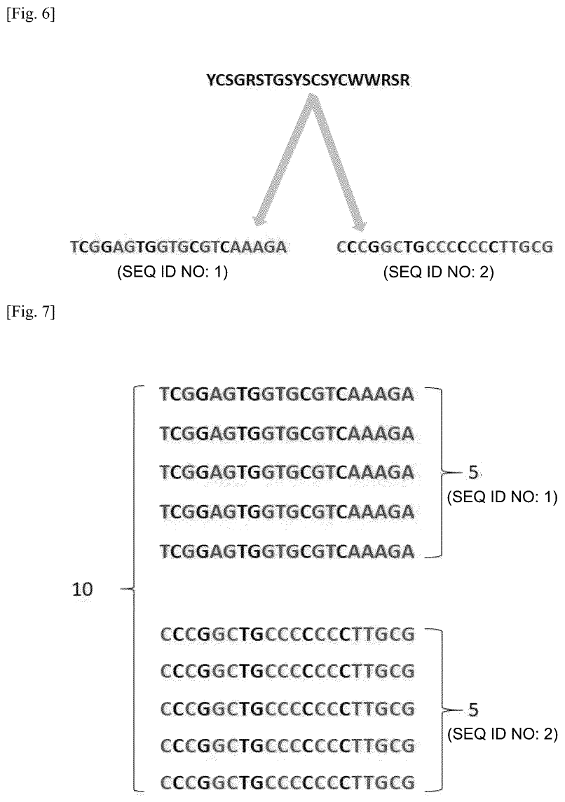

FIG. 6 is an exemplary diagram of a DNA strand produced when a D2 degenerate base is used.

Referring to FIG. 6, the sequence information converted into basic base values and degenerate base values can be confirmed in the same manner as illustrated in FIG. 5. Since the DNA strand is synthesized with A, G, C, and T, which are the actual basic bases, the DNA strand needs to change the sites occupied by the degenerate bases into basic bases. Thus, the processor can generate the sequence information “YCSGRSTGSYSCSYCWWRSR” as a DNA strand with “TCGGAGTGGTGCGTCAAAGA (SEQ ID NO: 1)” and “CCCGGCTGCCCCCCCTTGCG (SEQ ID NO: 2)”. Although FIG. 6 illustrates an example in which two DNA strands are generated for simplicity and convenience of understanding of the drawings, the generated DNA strands may be two or more.

In the next step s130, the processor may uniformly distribute the total number of DNA generated strands to be generated in the DNA synthesis step according to the type of DNA strands to be generated in the DNA synthesis step. As described above, a plurality of DNA strands may be generated to store a single binary data. For example, it is assumed that a total of 10 DNA strands are generated to store a single binary data. FIG. 7 is an example of 10 DNA strands distributed according to the type of DNA strands. Referring to FIG. 7, since the type of the DNA strand shown in FIG. 6 is two, it can be confirmed that 10 DNA strands are distributed at 5:5.

Hereinafter, a DNA decoding method according to the present specification will be described. The DNA decoding method according to the present specification presupposes that the binary data converted by the DNA encoding method according to the present specification is synthesized with a plurality of DNA strands. Meanwhile, the DNA decoding method according to the present specification may be implemented in the form of a computer program recorded on a recording medium readable by a computer by being prepared to perform each steps to be described below in a computer. When the DNA encoding method according to the present specification is implemented in the form of a computer program, each step may be executed by a processor.

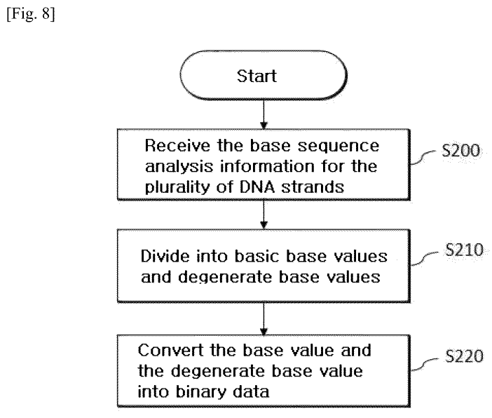

FIG. 8 is a flowchart of a DNA decoding method according to the present specification.

Referring to FIG. 8, first, in step s200, the processor may receive the base sequence analysis information for the plurality of DNA strands. The base sequence information may be output from a DNA analysis device that analyzes the base sequence of a DNA molecule. Referring again to FIG. 7, the processor may receive the base sequence analysis information for 10 DNA strands from the DNA analysis device.

In the next step s210, the processor can divide into basic base values and degenerate base values according to the base type for the same position in the plurality of DNA strands. In this case, the processor may be divided into basic base values and degenerate base values according to the ratio of base types to the same position in the plurality of DNA strands.

Referring again to FIG. 7, ten DNA strands where the second base is “C” in the plurality of DNA strands. In this case, the processor may be divided into basic base values because the second-digit base is one (100%) type. On the other hand, in the plurality of DNA strands, five DNA strands where the third base is “G”, and five DNA strands where “C” are. In this case, the processor may be divided into a degenerate base value because the third-digit base is two types (50%, respectively). In addition, the processor may distinguish that the third position is a degenerate base value “S” used by “G” and “C” bases. Through this process, the process shown in FIG. 6 can be reversed to obtain the sequence information “YCSGRSTGSYSCSYCWWRSR”.

On the other hand, an error may occur in the process of analyzing the base sequence for each DNA strand by the DNA analysis device. For example, as a result of analyzing the base at the second position in the ten DNA strands shown in FIG. 7, it is assumed that the “C” value is nine and the “G” value is one. First, the DNA encoding method according to the present specification can be uniformly distributed according to the type of DNA strands to be generated in the DNA synthesis step. Therefore, it is desirable to distribute the DNA strand at 5:5, but not 9:1. From this point of view, it is reasonable to judge “G” as an analysis error if “C” values are 9 and “G” values are 1 in 10 DNA strands. In order to cope with such an analysis error, according to an embodiment of the present specification, the processor may exclude a base type less than a preset reference number from a basic base value and a degenerate base value. The reference number may be variously set in consideration of the total number of DNA strands, the analysis error rate of the DNA analysis device, and various environmental factors of the DNA information storage system.

In the next step s220, the processor may convert the base value and the degenerate base value into binary data. This process is the opposite process of the process described above with reference to FIG. 5, so detailed descriptions are omitted.

The DNA information storage system according to the present specification may include a DNA encoding apparatus that executes the above-described DNA encoding method and a DNA decoding apparatus that executes the above-described DNA decoding method. In addition, the DNA information storage system according to the present specification may further include a DNA synthesizing device that synthesizes DNA according to the nucleotide sequence information output from the DNA encoding device and a DNA analyzing device that analyzes the nucleotide sequence of the DNA molecule and outputs the nucleotide sequence information.

Meanwhile, in the present specification, for convenience of understanding, the degenerate bases were described using only W, S, R, and Y, but it is obvious that the remaining degenerate bases M, K, B, V, H, D, and N can also be used. It is obvious that the notation and composition of each degenerate base can be varied, and the binary value corresponding to each degenerate base can also vary.

So far, the DNA encoding and decoding methods according to one embodiment of the present specification were examined. However, the DNA encoding according to an embodiment of the present specification matches one data value with one base value at 1:1. Therefore, the same bases in the base sequence can be converted to be placed repeatedly according to the binary data. If we look at “CCCGGCTGCCCCCCCTTGCG (SEQ ID NO: 2)” among the base sequences shown in FIG. 6, it can be confirmed that “C (cytosine)” is serially placed 7 times in the middle. According to Poon and MacGregor (1998) Biopolymers 45:427-434, it is mentioned that if G (guanine) is repeatedly synthesized by more than four, there is a problem of aggregation in the form of guanie tetraplex. Although the above academic data refers to a problem of G (guanine), it is not excluded that the same or similar problems do not occur for A (adenine), T (thymine), and C (cytosine). Therefore, in the storage of information using the DNA nucleotide sequence, a method of reducing the possibility of defects when the same bases are repeatedly synthesized in the process of synthesizing DNA is necessary.

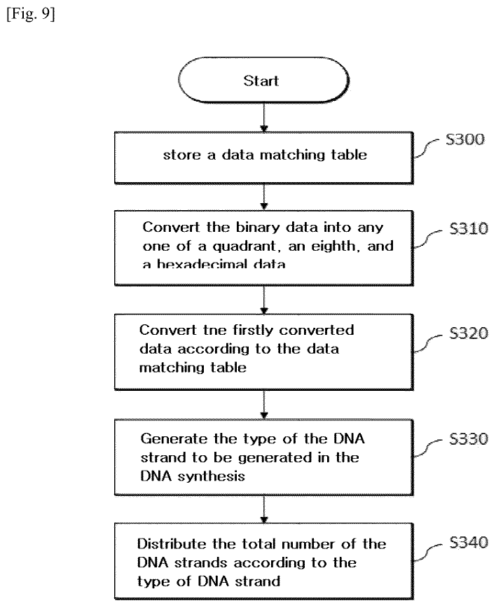

FIG. 9 is a flowchart of a DNA encoding method according to another embodiment of the present specification.

Referring to FIG. 9, first, in step s300, the processor may store a data transformation matching table that converts data into a base value. The data transformation matching table according to the present specification is a table that records the base values converted when converting the data value into a base sequence. The data transformation matching table according to the present specification has the purpose of preventing the same bases in the converted base sequence from being repeated, and may be composed of two base value combinations. To better understand the data transformation matching table according to the present specification, first, the two base value combinations will be described.

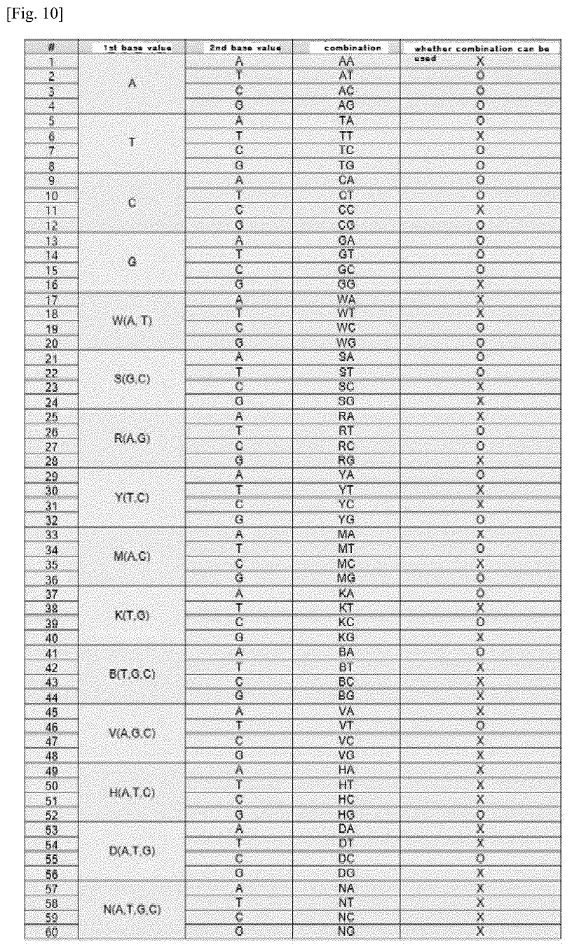

FIG. 10 is a table that indicates whether the two base value combinations overlap.

Referring to FIG. 10, it can be confirmed that the two base value combinations include at least one of the base value and the degenerate base value. As described above, the degenerate base value is at least two base value combinations among four basic bases, so the two base value combinations can be totally 60 base values. At this time, the base values first arranged in the two base value combinations were designated as the “first base value”, and the base values next arranged were designated as the “second base value”. In addition, the two base value combinations were designated as “x” in order not to be used when they overlap, and the two base value combinations were designated as “o” in order to be used when they do not overlap. As a result, the two base value combinations that can be used are totally 28 base values.

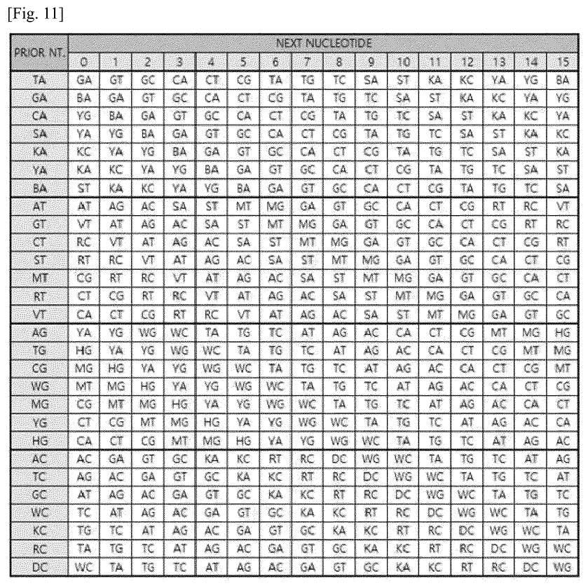

FIG. 11 is an example of the data transformation matching table according to an embodiment of the present specification.

Referring to FIG. 11, it can be confirmed that 28 base value combinations are displayed in the left column, and the data value is displayed in the top row. The data transformation matching table according to the present specification is characterized by consisting of transformation base values that match each data according to a preceding base value. For example, when the data value “0” is to be transformed, if the previously transformed two base value combinations are “TA,” it is to be transformed to “GA.” On the other hand, when the data value “0” is to be transformed, if the previously transformed two base value combinations are “GA,” it is to be transformed to “BA.” These data values are “0”, but the base values that are transformed can be changed according to the preceding base values.

The preceding base value and the transformation base value of the data transformation matching table according to the present specification are combinations of two base values including at least one of the base values among given basic base values and degenerate base values. In particular, the transformation base value of the data transformation matching table may be configured to have a second base value different among the preceding base values and a first base value of the transformation base value. Through this, it can be prevent the same the same bases parts overlap from being disposed when the data is converted into a base sequence.

Referring again to FIG. 9, in step s310, the processor may first convert the binary data into any one of a quadrant, an eighth, and a hexadecimal data. When converting binary data consisting of “0” and “1”, it can be converted into quaternary data if it is converted by grouping two bits by two bits. Likewise, if binary data is bundled by 4 bits, it can be converted into hexadecimal data if binary data is bundled by 8 bits. As such, the processor converts the binary data into 4, 8 or 16 binary data is referred to herein as “primary conversion”.

In the next step s320, the processor may secondarily convert the firstarily converted data into sequence information using basic base values and degenerate base values according to the data conversion matching table. For convenience of understanding, it will be explained by way of example.

It will be assumed that “DNA” is the original information to be converted. The three word characters are expressed as binary data according to utf-8 as follows.
[01000100 01001110 01000001]₍₂₎

The binary data is converted again into a hexadecimal number, as follows.
[44 4e 41]₍₁₆₎

The secondary converted data will be converted according to a data conversion matching table.

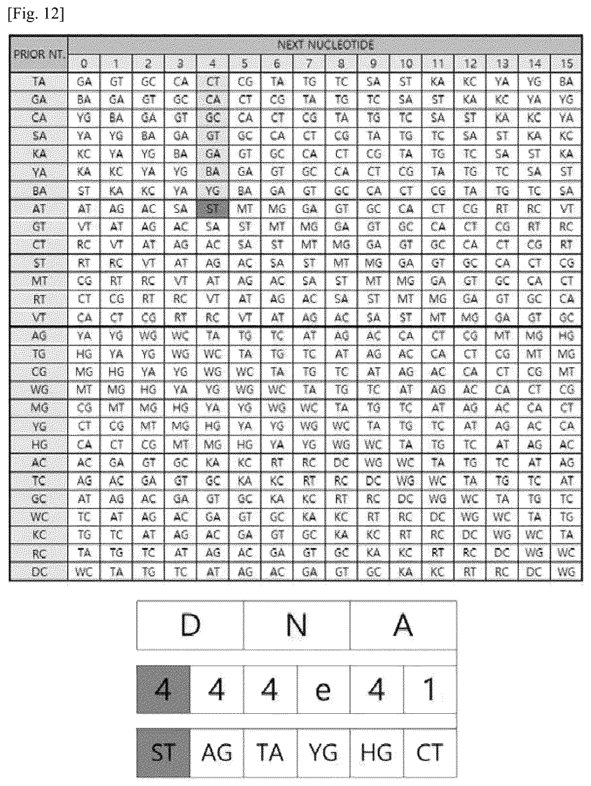

FIGS. 12 to 15 are examples of secondary transformations.

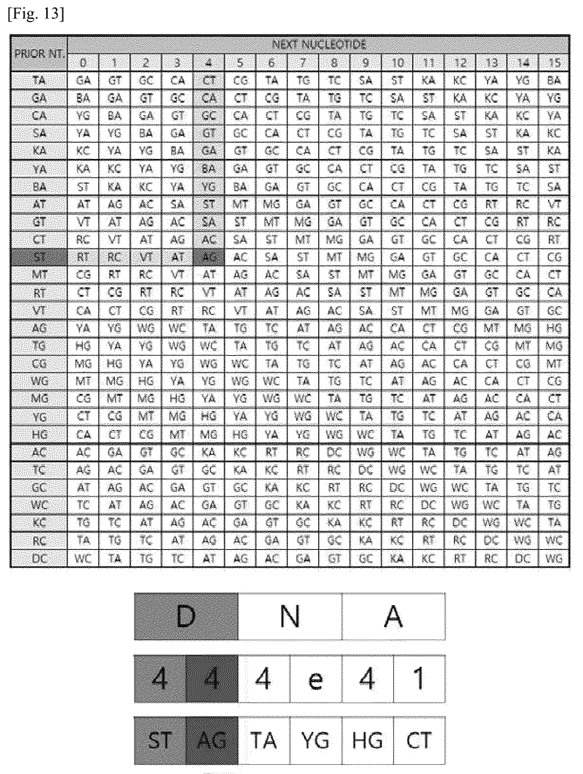

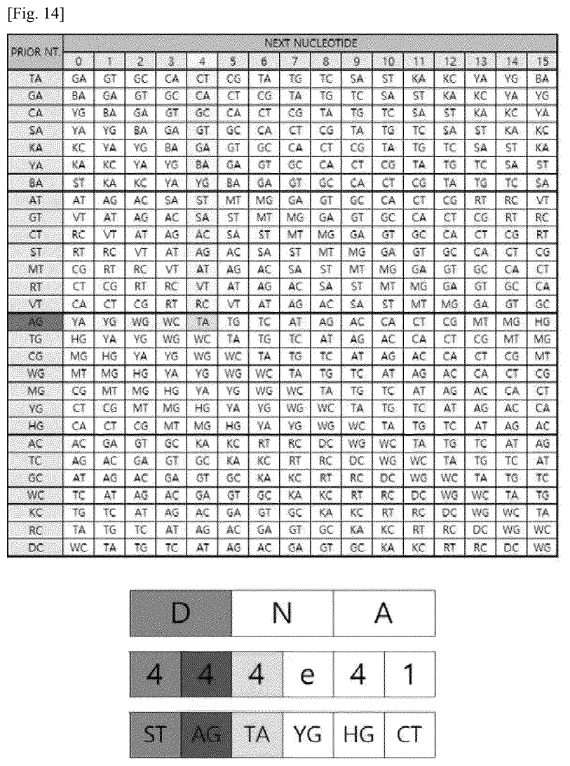

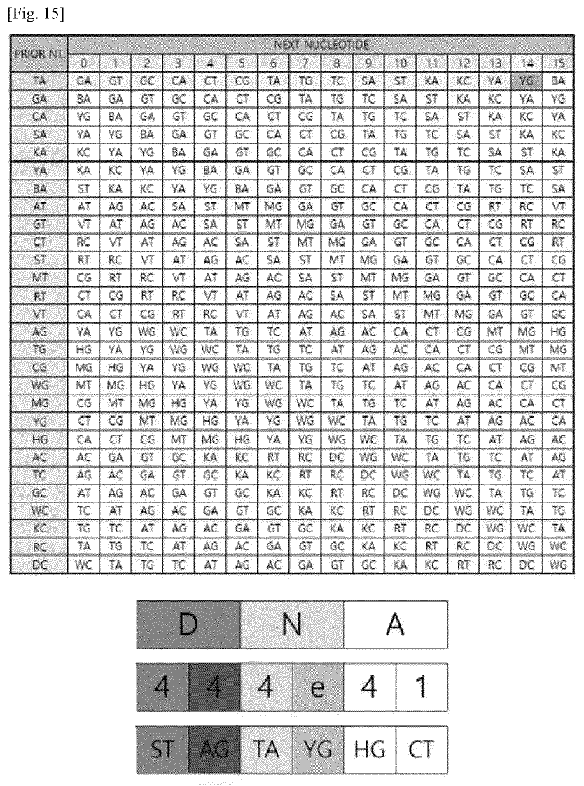

Referring to FIG. 12, it can be confirmed that the first data “4” has been converted into a “ST” base value. In this way, if there is no prior base value, any base value among the base values corresponding to data “4” may be selected. Referring to FIG. 13, it can be confirmed that the second data “4” has been converted into the base value “AG” because the previous base value is “ST”. Referring to FIG. 14, it can be confirmed that the third data “4” has been converted into a base value of “TA” because the previous base value is “AG”. Referring to FIG. 15, it can be confirmed that the fourth data “E” has been converted into a base value of “YG” because the previous base value is “TA”. After that, the fifth data “4” may be converted into a “HG” base value, and the sixth data “1” may be converted into a “CT” base value.

As a result, the second-transformed sequence information may be “STAGTAYGHGCT”. Looking at the secondary conversion process, it can be seen that the first data, the second data, the third data, and the fifth data are all “4” but are all converted into different base values.

Thereafter, steps s330 and s340 are the same as steps s120 and s130 of FIG. 3 above, and thus repeated explanations are omitted. In this case, the second-transformed sequence information may be synthesized as the DNA strand “(G/C)TAGTA(T/C)G(A/T/C)GCT (SEQ ID NO: 3)” composed of the actual basic base, and it can be confirmed that the same base overlap does not occur between adjacent bases even if the degenerate base site is replaced with the basic base.

Meanwhile, the data transformation matching table according to the present specification is an example for preventing duplication, and the data and base values do not necessarily coincide as shown in the drawings.

The DNA information storage system according to the present specification may include a DNA encoding apparatus that executes the above-described DNA encoding method and a DNA decoding apparatus that executes the above-described DNA decoding method. In addition, the DNA information storage system according to the present specification may further include a DNA synthesizing apparatus that synthesizes DNA according to the nucleotide sequence information output from the DNA encoding apparatus and a DNA analyzing apparatus that analyzes the nucleotide sequence of the DNA molecule and outputs the nucleotide sequence information.

Meanwhile, in the present specification, for convenience of understanding, degenerate bases were described using W, S, R, and Y, but it is obvious that the remaining degenerate bases M, K, B, V, H, D, and N may also be used. It is obvious that the notation and configuration of each degenerate base can be varied, and the binary values corresponding to each degenerate base can also be varied.

The above-described computer program may include code coded in computer language such as C/C++, C#, JAVA, Python, and machine language, in which the processor (CPU) of the computer can read through the device interface, so that the computer reads the program and executes the above-described methods implemented in the program. Such code may include functional code related to functions defining the necessary functions for executing the above-described methods, and may include execution procedure related control code necessary for the processor of the computer to execute the above-described functions according to a predetermined procedure. In addition, the code may further include memory reference related code for additional information or media necessary for the processor of the computer to execute the above-described functions to be referenced at which location (address border) of the internal or external memory of the computer. In addition, in the case where the processor of the computer needs to communicate with any other computer or server at a remote location to execute the above-described functions, the code may further include communication related code for how to communicate with any other computer or server at a remote location using the communication module of the computer, and which information or media should be transmitted and received at the time of communication.

The medium to be stored means a medium that stores data semi-permanently and can be read by a device, rather than a medium that stores data for a short moment, such as a register, cache, and memory. Specifically, examples of the medium to be stored include ROM, RAM, CD-ROM, magnetic tape, floppy disk, and optical data storage, but are not limited thereto. That is, the program may be stored in various recording media on various servers that can be accessed by the computer or various recording media on the computer of the user. In addition, the medium may be distributed to a computer system connected to a network, and may be stored with computer readable code in a distributed manner.

Hereinafter, a single-strand DNA structure analyzing apparatus according to another embodiment of the present invention will be described in detail.

An exemplary embodiment of the present invention provides a single-stranded DNA structure analysis apparatus comprising: a DNA data storage unit that stores a single-stranded DNA structure; a DNA data receiving unit that receives the analyzed single-stranded DNA structure; a separation plate that includes a nanochannel that communicates with the DNA data storage unit; and a first capacitor electrode and a second capacitor electrode that are provided to be spaced apart by the nanochannel and electrically connected via a capacitance sensing unit.

The single-stranded DNA structure analysis apparatus according to the present invention has the advantage of effectively storing, searching, and reading data of a very large capacity compared to existing recording media such as HDD and SSD by using the single-stranded DNA structure. Hereinafter, each component of the single-stranded DNA structure analysis device will be described in detail.

### DNA Data Storage

The DNA data storage unit in the single-stranded DNA structure analysis device may be a place for storing a single-stranded DNA structure encoded with predetermined data. Specifically, the DNA data storage unit may be a place in which the single-stranded DNA structure is stored by applying a negative voltage or a ground voltage in the electrolyte solution.

According to an exemplary embodiment of the present invention, the DNA data storage unit may further include a plurality of DNA control electrodes, and the DNA control electrodes may detach single-stranded DNA by applying a positive voltage or a negative voltage. Specifically, the DNA control electrode may apply a positive voltage to attach a single-stranded DNA structure when data is to be stored. In addition, the DNA control electrode may detach the single-stranded DNA structure by applying a negative voltage when data read is necessary. The plurality of DNA control electrodes may be provided to control each of the plurality of single-stranded DNA structures encoded with different information. That is, by individually controlling a plurality of single-stranded DNA structures using a plurality of DNA control electrodes, a plurality of information can be effectively stored and searched.

According to an embodiment of the present invention, the DNA data storage unit may include an electrolyte solution. Specifically, the DNA data storage unit may be filled with an electrolyte solution such as potassium chloride and/or sodium chloride, and the single-stranded DNA structure detached by the current applied to the first/second traction electrode to be described later may be drawn to the DNA data storage unit via the electrolyte solution. The electrolyte solution may be applied without limitation as long as it is not only the electrolyte solution described above but also an electrolyte solution capable of performing the same function.

### DNA Data Receptacle

The DNA data receiving part in the single-stranded DNA structure analysis device is a place to receive the single-stranded DNA structure drawn through the nanochannel, and the single-stranded DNA structure reaching the DNA data receiving part may not be utilized any longer as an information medium. Specifically, the single-stranded DNA structure detached from the control electrode of the DNA data storage unit passes through the nanochannel, and the encoded base sequence of data may be analyzed to perform data read, and the single-stranded DNA completed to read data may be disposed from the DNA data storage unit.

According to an exemplary embodiment of the present invention, the DNA data receiver may include an electrolyte solution. As described above, the single stranded DNA structure may move via an electrolyte solution, and the DNA data storage portion and the DNA data receiving portion may be filled with an electrolyte solution. That is, the DNA data storage unit, the DNA data receiver, and the separator may be connected with the same electrolyte solution.

### Separator

The separator in the single-stranded DNA structure analysis device may include a nanochannel. Furthermore, the separator may be provided to be spaced apart by the nanochannel, and include a first capacitor electrode and a second capacitor electrode electrically connected via the capacitance sensing unit, and the capacitance that varies according to the intrinsic permittivity of individual bases of the single-stranded DNA structure passing through the nanochannel may be measured by the sensing unit, and the data encoded in the single-stranded DNA structure may be decoded using the measured capacitance.

According to one embodiment of the present invention, the capacitance sensing unit may include an alternating current source within a frequency range of 20 Hz to 2 MHz. The capacitance sensing unit may measure a fine change in capacitance in the nanochannel by applying an alternating current with a high frequency in the frequency range of 20 Hz to 2 MHz.

According to one embodiment of the present invention, the capacitance sensing unit may analyze the base sequence of the single-stranded DNA structure by sensing a change in capacitance that varies according to the intrinsic permittivity of each of adenine (A), thymine (T), guanine (G), and cytosine (C) in the single-stranded DNA structure passing through the nanochannel. Specifically, the nanochannel may be filled with an electrolyte solution, the capacitance according to the permittivity of the electrolyte solution may be recognized as a basic value, and the capacitance that varies according to the intrinsic permittivity of individual bases of the single-stranded DNA structure passing through the nanochannel may be sequentially sensed, and the base sequence of the single-stranded DNA structure may be analyzed using the sensed capacitance.

According to one embodiment of the present invention, the data processing unit may further include a data processing unit that performs decoding using the information obtained by the capacitance sensing unit. The data processing unit may perform data reading by decoding the information encoded according to the base sequence of the single-stranded DNA structure. The decoding performed by the data processing unit may be performed by applying a method known in the art.

According to one embodiment of the present invention, the diameter of the nanochannel may be in the range of 0.5 nm to 5 nm. Specifically, the diameter of the nanochannel may be in the range of 0.5 nm to 3 nm, 0.5 nm to 2 nm, or 0.5 nm to 1.5 nm. The diameter of the nanochannel may mean the maximum length in the cross-section of the nanochannel. When the diameter of the nanochannel is in the range, the single-stranded DNA structure moving from the DNA data storage unit may pass through the nanochannel one by one, thereby increasing the accuracy of data analysis.

According to one embodiment of the present invention, the nanochannel may be provided in the separator, and each nanochannel may pass through the single-stranded DNA structure one by one, thereby analyzing the information encoded in the single-stranded DNA structure passing through the single-stranded single-stranded DNA structure.

### First/Second Traction Electrode

The single-stranded DNA structure analysis device may further include a first DNA traction electrode provided in the DNA data storage unit, and a second DNA traction electrode provided in the DNA data receiver. The first traction electrode and the second traction electrode may be electrodes each containing a material selected from the group consisting of Cu, Pt, Ag, Au, W, Ti, Al, TN, ITO, and polysilicon.

According to an exemplary embodiment of the present invention, the first DNA traction electrode and the second DNA traction electrode may be electrically connected through a current sensing unit. According to an exemplary embodiment of the present invention, by applying a bias to the second DNA traction electrode, a single-stranded DNA structure of the DNA data storage unit may be moved to the DNA data storage unit through the nanochannel. Specifically, the first traction electrode may be provided on the top of the DNA storage unit, and the second traction electrode may be provided on the bottom of the DNA data receiver. Furthermore, by applying a positive voltage to the second traction electrode, it is possible to pull the single-stranded DNA structure floating in the electrolyte solution.

According to an exemplary embodiment of the present invention, the current sensing unit may sense a change in current that is fluctuated by the molecular size of adenine (A), thymine (T), guanine (G), and cytosine (C) in the single-stranded DNA structure passing through the nanochannel, and analyze the nucleotide sequence of the single-stranded DNA structure. Specifically, the current sensing unit may measure that ions of an electrolyte solution move from the DNA data storage unit to the DNA data receiving unit, and may sense a minute change in current generated by the single-stranded DNA structure passing through the nanochannel to interfere with the ion flow. More specifically, the difference in the degree of interference in ion flow in the nanochannel can be analyzed according to the size of each base in the single-stranded DNA structure passing through the nanochannel, and the base sequence of the single-stranded DNA structure can be analyzed using the above-described data processing unit etc.

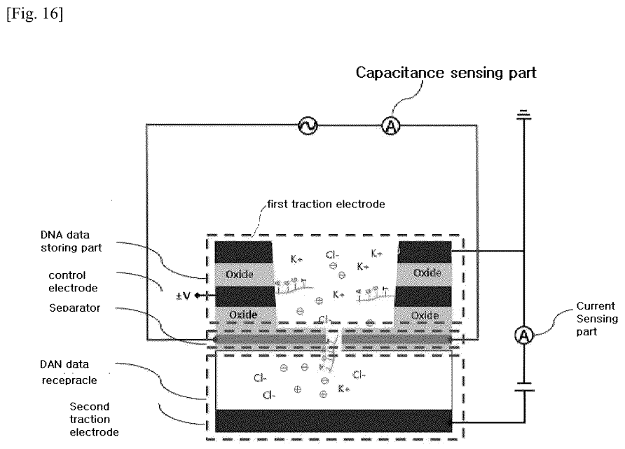

FIG. 16 schematically shows a single-stranded DNA structure analysis apparatus according to an embodiment of the present invention. Specifically, FIG. 16 is a schematic diagram of a single-stranded DNA structure provided with a DNA data storage unit and a DNA data receiving unit with a separator interposed therebetween, and detached from the ions and control electrodes in the electrolyte solution by the first and second traction electrodes passing through the nanochannel. However, the single-stranded DNA structure analysis apparatus according to the present invention is not limited to the structure of FIG. 16, and some components may be excluded or added.

Another embodiment of the present invention provides a single-stranded DNA structure analysis method using the single-stranded DNA structure analysis apparatus, comprising: (a) receiving an electrolyte solution in the DNA data storage unit and the DNA data receiver of the single-stranded DNA structure analysis apparatus; (b) moving the single-stranded DNA structure of the DNA data storage unit to the DNA data receiver through the nanochannel; and (c) analyzing the base sequence of the single-stranded DNA structure passing through the nanochannel using the base monitoring current value Im of the single-stranded DNA structure according to Equation 1 below.

$${I}_{m} = \frac{{C}_{b}}{{C}_{s}} \cdot \frac{d V}{d t} \qquad equation 1$$

In Equation 1, Cs is the capacitance value in a state in which the nanochannel is filled with the electrolyte solution, Cb is the capacitance value according to the intrinsic permittivity of each base of the single-stranded DNA structure passing through the nanochannel, and V is the voltage.

The single-stranded DNA structure analysis method according to the present invention may be to analyze single-stranded DNA structure using the components of the single-stranded DNA structure analysis apparatus described above, and the detailed description of the single-stranded DNA structure analysis apparatus described above is integrated into the single-stranded DNA structure analysis method. Hereinafter, each step of the single-stranded DNA structure analysis method will be described in detail.

### Step (a): Receiving an Electrolyte Solution.

The step (a) may include accommodating an electrolyte solution as a medium that allows the single-stranded DNA structure to move in the DNA data storage and the DNA data accommodation part. Since the DNA data storage unit and the DNA data accommodation unit are provided with a separation plate including the aforementioned nanochannel, the DNA data storage unit, the DNA data accommodation unit, and the nanochannel may be filled with the electrolyte solution. The electrolyte solution may be a solution containing salts from which cations and anions are dissociated in the solution, for example, a potassium chloride aqueous solution and/or a sodium chloride aqueous solution. However, the present invention is not limited thereto, and it can be applied without limitation as long as it is an electrolyte solution capable of performing the same function.

According to an exemplary embodiment of the present invention, it may further include (al) a step of applying a negative voltage to at least one of a plurality of DNA control electrodes provided in the DNA data storage unit to separate a single-stranded DNA structure from the DNA control electrode. In this case, the magnitude of the negative voltage applied to the DNA control electrode may be 0 v to −10 v. As described above, the DNA control electrodes may be provided in plurality, and each of them may store a single-stranded DNA structure in which different information is encoded.

### Step (b): Moving the Single-Stranded DNA Construct of the DNA Data Storage Unit to the DNA Data Receiver.

The step (b) may be to move the single-stranded DNA structure stored in the DNA data storage unit through the nanochannel of the separation plate to the DNA data receiver.

According to an exemplary embodiment of the present invention, the DNA data storage unit may include a first DNA traction electrode, and the DNA data storage unit may include a second DNA traction electrode. The first DNA traction electrode and the second DNA traction electrode may be electrically connected through ion movement of the electrolyte solution. Furthermore, when a current is applied to the first DNA traction electrode and the second DNA traction electrode, a voltage gradient occurs in the electrolyte solution to move ions in the electrolyte solution, and furthermore, a single-stranded DNA structure stored in the DNA data storage unit may be moved. Specifically, in step (b), a bias may be applied to the second DNA traction electrode provided in the DNA data receiver to move single-stranded DNA of the DNA data storage unit through the nanochannel to the DNA data receiver. In this case, the magnitude of the bias applied to the second DNA traction electrode may be in the range of 0.5 v to 3 v.

According to an exemplary embodiment of the present invention, the first DNA traction electrode and the second DNA traction electrode may be electrically connected via a current sensing unit, and as described above, the current sensing unit may sense a minute change in current generated by the single-stranded DNA structure passing through the nanochannel to interfere with the ion flow.

Specifically, according to an exemplary embodiment of the present invention, it may further include (b1) analyzing the base sequence of the single-stranded DNA construct by detecting a change in current that is fluctuated by the molecular size of adenine (A), thymine (T), guanine (G), and cytosine (C) in the single-stranded DNA construct passing through the nanochannel. More specifically, the current sensing unit may analyze the difference in the degree of interference of ion flow in the nanochannel according to the size of each base in the single-stranded DNA structure passing through the nanochannel, and may use this to analyze the base sequence of the single-stranded DNA structure using the aforementioned data processing unit.

### (c): Analyzing the Base Sequence of the Single-Stranded DNA Construct Passing Through the Nanochannel.

The step (c) may be analyzing the base sequence of the single-stranded DNA construct passing through the nanochannel by using the base-specific monitoring current value Im of the single-stranded DNA construct according to the following Equation 1.

$${I}_{m} = \frac{{C}_{b}}{{C}_{s}} \cdot \frac{d V}{d t} \qquad Equation 1$$

In Equation 1, Cs is the capacitance value in the state in which the nanochannel is filled with the electrolyte solution, Cb is the capacitance value according to the intrinsic permittivity of each base of the single-stranded DNA construct passing through the nanochannel, and V is the voltage.

The single-stranded DNA construct consists of a base of adenine (a), thymine (t), guanine (g), and cytosine (c), and each of these bases has the intrinsic permittivity, and may represent the following capacitance values:

$${C}_{A} = {ε}_{A} {ε}_{0} A / d ,$$

such that

$${C}_{T} = {ε}_{T} {ε}_{0} A / d ,$$

such that

$${C}_{G} = {ε}_{G} {ε}_{0} A / d ,$$

such that

$${C}_{C} = {ε}_{C} {ε}_{0} A / d .$$

such that

Here, Ca, CT, CG, and CC are the capacitance values of adenine (A), thymine (T), guanine (G), and cytosine (C), respectively, A is the electrode area, and d is the electrode spacing. In the state in which the single-stranded DNA construct does not pass, the dielectric material present between the first/second capacitance electrodes becomes the electrolyte solution, and at this time, the same current value will be monitored when constantly applying the change in voltage at a certain time.

In addition, when the single-stranded DNA construct passes through the nanochannel, the capacitance value varies by each base having the intrinsic permittivity, so that the base-specific monitoring current value Im may be monitored differently over time, and by analyzing the capacitance value, the base sequence of the single-stranded DNA construct may be accurately measured, and the information stored thereto may be decoded. According to an embodiment of the present invention, the step (c) may use an alternating current within a frequency range of 20 Hz to 2 MHz. The step (c) may sense the current flowing through the AC voltage and measure L (inductor), C (capacitor), and R (resistor).

While a general impedance analyzer uses a very wide frequency range of 20 Hz to 1 GHz, the frequency range in the step (c) of the present invention may be used to measure the more accurate capacitance value by applying a limited range of 20 Hz to 2 MHz.

The capacitance variation may be measured by using the first capacitor electrode and the second capacitor electrode provided to be spaced apart by the nanochannel and electrically connected via the capacitance sensing unit, and the capacitance varying according to the intrinsic permittivity of each base of the single-stranded DNA construct passing through the nanochannel may be measured by the sensing unit, and the data encoded in the single-stranded DNA construct may be decoded by using the capacitance varying according to the intrinsic permittivity of each base of the single-stranded DNA construct passing through the nanochannel. Specifically, the nanochannel may be filled with the electrolyte solution, the capacitance according to the permittivity of the electrolyte solution may be recognized as a default value, and the capacitance varying according to the intrinsic permittivity of each base of the single-stranded DNA construct passing through the nanochannel may be sequentially sensed, and the base sequence of the single-stranded DNA construct may be analyzed by using the capacitance varying according to the intrinsic permittivity of each base of the single-stranded DNA passing through the nanochannel. The analysis of the base sequence may be performed using a data processing unit that performs decoding using information obtained from the capacitance sensing unit, and the decoding performed by the data processing unit may be performed by applying methods known in the art.

The single-stranded DNA structure analysis method according to the present invention may analyze single-stranded DNA structure primarily using information obtained from the current sensing unit, and further verify the analysis contents using information obtained from the capacitance sensing unit to enable more accurate analysis.

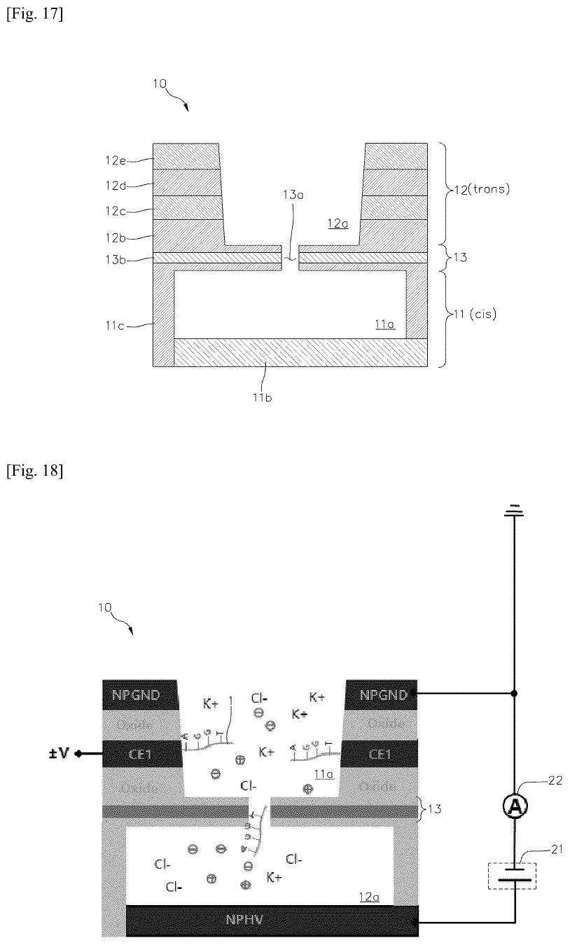

FIG. 17 is a cross-sectional view schematically showing a semiconductor-based DNA chip according to another embodiment of the present invention.

Referring to FIG. 17, the DNA chip 10 according to an embodiment of the present invention includes a silicon-based nanopore membrane 13 including first and second fluid compartments 11 and 12 filled with electrolyte and a single nanopore 13 a fluidly connecting the first and second fluid compartments 11 and 12 to each other.

The first and second fluid compartments 11 and 12 include first and second fluid reservoirs 11a and 12a filled with conductive electrolyte. The first fluid reservoir 11a communicates with the second fluid reservoir 12a of the second fluid compartment 12 through the single nanopore 13a of the nanopore membrane 13, so that the fluids filled in the first and second fluid reservoirs 11a are connected to each other through the single nanopore 13a.

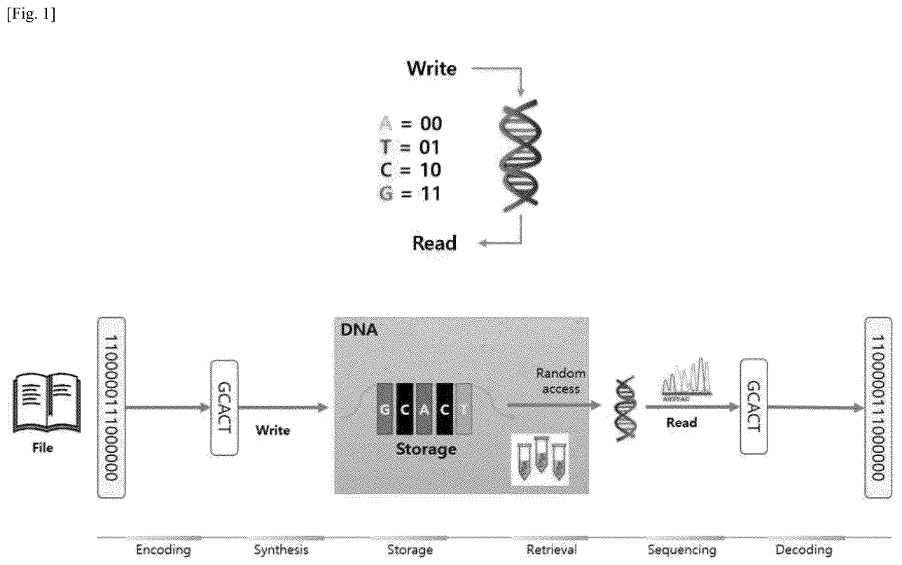

As shown in FIG. 1, the first fluid reservoir 11a may be partitioned into a cavity shape by the nanopore film 13 formed on the top, the electrode layer 11b formed on the bottom, and the insulating film 11c formed on the side. The internal volume of the first fluid reservoir 11a may be larger or smaller than the internal volume of the second fluid reservoir 12a. Or it may be the same. The insulating layer 11c forming the sidewall of the first fluid reservoir 11a may be formed of a silicon oxide layer (SiO2). The electrode layer 11 b is a conductive layer for applying voltage to the first fluid reservoir 11 a, and may be made of any one metal selected from metals such as tungsten (W), titanium (Ti), titanium nitride (TiN), aluminum (Al), copper (Cu), platinum (Pt), silver (Ag), or gold (Au). It may also be made of an alloy or metal oxide film in which a double metal is mixed.

The second fluid reservoir 12a is formed to have an internal volume of a predetermined size in the center of the second fluid partition 12. The second fluid reservoir 12a may be partitioned by the nanopore film 13 formed in the lower portion, the insulating films 12b and 12d and the electrode layers 12c and 12e stacked on the side portions. The insulating films 12b and 12d are films for electrically separating the nanopore film 13, the electrode layer 12c, and the electrode layers 12c and 12e, and may be made of a dielectric film having a predetermined dielectric constant. For example, the insulating films 12b and 12d may be made of silicon oxide (SiO2) having the same dielectric constant. Each of the electrode layers 12c and 12e is a conductive layer to which a voltage is applied to the second fluid compartment 12, and may be made of any one metal selected from among metals such as tungsten (W), titanium (Ti), titanium nitride (TiN), aluminum (Al), copper (Cu), platinum (Pt), silver (Ag), or gold (Au). It may also be made of an alloy or metal oxide film in which a double metal is mixed.

The nanopore membrane 13 has a single nanopore 13a that communicates the first and second fluid reservoirs 11a and 12a with each other. The single nanopore 13a is a passage through which ions of an electrolyte solution pass, and may function as a channel connecting the electrolyte solution stored in the first and second fluid reservoirs 11a and 12a, respectively. When a voltage is applied, the ions of the electrolyte solution filled in the second fluid reservoir 12a move to the first fluid reservoir 11a through a single nanopore 13a according to the principle of electrophoresis. When the single strand DNA stored in the second fluid reservoir 12a interferes with the charge flow (current flow) of the electrolyte solution in the single nanopore 13a in the process of passing through the single nanopore 13a, the magnitude of the current changes depending on the sequence of the single strand DNA having different molecular sizes.

The diameter of the single nanopore 13a needs to be controlled within the range of approximately 1 to 5 nm to detect micromolecules such as DNA. For example, when the diameter of the single nanopore 13a is large, the passage rate of the analyte passing through the single nanopore 13a is too fast, so there may be a limit to show high consistency in detecting micromolecules such as single stranded DNA. Accordingly, in order to detect and analyze a micromolecule such as DNA, a technique of finely adjusting the diameter of a single nanopore 13a is more important than anything. In an embodiment of the present invention, the diameter of the single nanopore 13a can be finely adjusted by combining semiconductor technology.

In an embodiment of the present invention, as shown in FIG. 17, a nanopore film 13 is formed using a silicon-based insulating film. The silicon-based insulating film enables selective etching in semiconductor manufacturing technology, and such manufacturing technology can provide the diameter of the single nanopore 13a to be unadjustable. For silicon-based insulating films, for example, inorganic materials can be used. Preferably, a silicon nitride (Si3N4) can be used.

FIG. 18 is a cross-sectional view schematically showing a method of driving a semiconductor substrate DNA chip according to an embodiment of the present invention, and is a cross-sectional view schematically showing a method of storing and reading data.

As shown in FIG. 18, in the semiconductor-based DNA chip 10 according to the embodiment of the present invention, one electrode layer 11b is formed in the first fluid partition 11 to apply voltage to the DNA chip 10 to induce the flow of charges of the conductive electrolyte solution, and two electrode layers 12c and 12e are formed in the second fluid partition 12. The number of electrode layers formed in the second fluid partition 12 may be expanded to three or more, thereby increasing the amount of data stored in the DNA chip.

A bias voltage of a predetermined magnitude is applied to the electrode layers 11b formed in the first fluid partition 11 and the electrode layers 12c and 12e formed in the second fluid partition 12. Depending on the magnitude of the bias voltage, data storage and read operations for the DNA chip 10 are performed.

In the following description of the embodiment of the present invention, the electrode layer 12c is referred to as a ‘control electrode (CE, Control Electrode)’, the electrode layer 12e is referred to as a ‘source electrode’, and the electrode layer 11b is referred to as a ‘drain electrode’ according to the characteristics (size) and functions of the voltage applied to each electrode layer for convenience of explanation. However, the scope of the technical features proposed in the embodiment of the present invention is not limited by these terms.

As shown in FIG. 18, a bias voltage (+V) of a predetermined magnitude is applied to the control electrode (CE1) to attach and fix the single stranded DNA (1) stored in the second fluid reservoir (12a) to the control electrode (CE1), or to drop off it. The source electrode NPGND and the drain electrode NPHV are each applied with a bias voltage to induce the flow of current by the charge of the electrolyte solution filled in the second fluid reservoir 12a flowing to the first fluid reservoir 11a through the single nanopore 13a. A bias voltage corresponding to a nanopore ground voltage may be applied to the source electrode NPGND, and a bias voltage corresponding to a nanopore high voltage may be applied to the drain electrode NPHV. The bias voltage applied to the control electrode CE1, the source electrode NPGND, and the drain electrode NPHV may be a direct current (DC) voltage.

The data storage operation of the DNA chip 10 may be defined as an operation in which the single strand DNA 1 is attached to the control electrode CE1 in the second fluid storage 12a by the bias voltage applied to the control electrode CE1, and the read operation may be defined as an operation in which a change in the current magnitude in the first and second fluid storages 11a and 12a is sensed through the current sensor 22 by the bias voltage applied to the drain electrode NPHV and the source electrode NPGND.

For example, when a positive voltage between 0.0 V and 10 V is applied to the control electrode CE1, the single strand DNA 1 synthesized with DNA bases (150-200 strands of DNA with bases) is attached to the control electrode CE1, and the attached state in which the single strand DNA 1 is drawn to the bias voltage applied to the control electrode CE1 is a state in which data is stored.

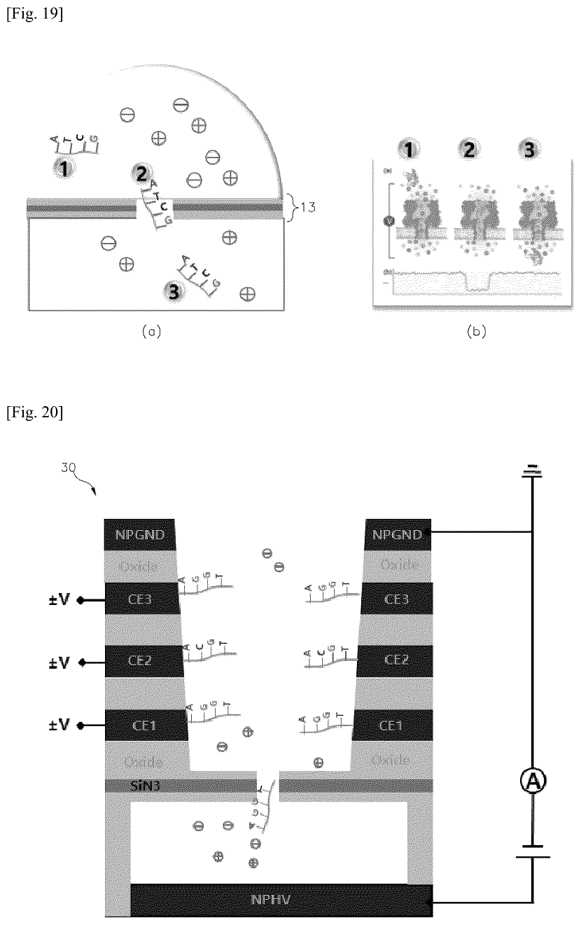

FIG. 19 is a conceptual diagram illustrating the data read operation according to the present invention, (a) shows DNA sequencing during the read operation, and (b) shows the change in the current during the read operation.

Referring to FIGS. 18 and 19, the read operation of the data (single strand DNA) stored in the second fluid storage 12a may be performed in the following manner. A negative voltage between 0.0 V and −10 V is applied to the control electrode CE1, and the single strand DNA 1 attached to the control electrode CE1 is detached from the control electrode CE1 and is separated from the control electrode CE1. Thereafter, a bias voltage between 0.5 V and 3 V is applied to the drain electrode NPHV through the voltage source 21 (direct current), and a ground voltage GND is applied to the source electrode NPGND. The electrolyte filled in the first and second fluid storages 11a and 12a, for example, the KCl electrolyte, is drawn to the source electrode NPGND by the bias condition, and the current flows in the first and second fluid storages 11a and 12a while the Cl− ion is drawn to the drain electrode NPHV in contrast to the K+. The flow of the current is sensed through the current sensor 22 and monitored.

When a bias voltage between 0.5 V and 3 V is applied to the drain electrode NPHV during the data read operation, and a ground voltage is applied to the source electrode NPGND, as shown in (a) of FIG. 3, the single strand DNA 1 present in the first fluid storage 11a is detached from the control electrode CE1 and then moved to the first fluid storage 11a through the single nanopore 13a a along with the charge of the electrolyte. For example, in the process of the single strand DNA 1 through the single nanopore 13a, the DNA bases adenine (A), thymine (T), cytosine (C), and guanine (G) block the single nanopore 13a to prevent the charge flow of the electrolyte. Due to the interruption of the charge flow of the electrolyte, the magnitude of the current is reduced differently as shown in (b) of FIG. 19. The degree of the interruption of the charge flow of the electrolyte varies depending on the molecular size of the DNA base. As a result, the magnitude of the current flowing in the first and second fluid reservoirs 11a and 12a due to the flow of the electrolyte changes in response to the molecular size of the DNA base, so that the reading operation of the data can be realized.

FIG. 20 is a cross-sectional view schematically illustrating a semiconductor-based DNA chip according to another embodiment of the present invention.

Referring to FIG. 20, the semiconductor-based DNA chip 30 according to another embodiment of the present invention presents an extended structure in the DNA chip 10, which is a basic unit structure shown in FIG. 18 (a structure in which one control electrode is configured). The extended DNA chip 30 may increase the data storage capacity.

Although the DNA chip 10 having the basic unit structure shown in FIG. 18 includes one control electrode CE1 in the second fluid compartment 12, the extended DNA chip 30 has a structure in which two or more control electrodes are stacked between the nanopore film 13 and the source electrode NPGND. That is, the extended DNA chip 30 may be formed with a plurality of control electrodes CE1 to CE3 in the second dielectric compartment. An insulating film (e.g., a silicon oxide film) may be formed between the plurality of control electrodes CE1 to CE3 and may be separated from each other. Each of the plurality of control electrodes CE1 to CE3 may be connected to an individual metal line (an address line function), and may receive a bias voltage, thereby addressing each control electrode. Other structures may be formed in the same structure as the DNA chip having the basic unit shown in FIG. 2.

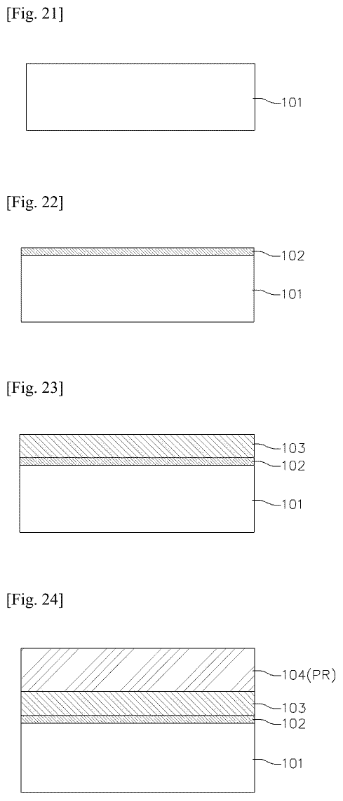

FIGS. 21 to 44 are process diagrams illustrating a method of manufacturing a semiconductor-based DNA chip according to an embodiment of the present invention, and are cross-sectional views schematically illustrating portions for convenience of explanation.

Referring to FIG. 21, a base substrate 101 is first prepared. As the base substrate 101, a silicon substrate may be used.

Subsequently, as shown in FIG. 22, an electrode layer 102 (hereinafter, referred to as a lower electrode layer) is formed on the upper portion of the base substrate 101 using a metal having conductivity. The lower electrode layer 102 may be an electrode corresponding to the drain electrode nphv shown in FIG. 18. The lower electrode layer 102 may be formed by any one of Chemical Vapor Deposition (CVD), Physical Vapor Deposition (PVD), sputter, or Atomic Layer Deposition (ALD). As the material, any one of metals such as tungsten, titanium (Ti), titanium nitride film (TiN), aluminum (Al), copper (Cu), platinum (Pt), silver (Ag), or gold (Au) may be formed. In addition, the lower electrode layer 102 may be formed of a single metal or an alloy having conductivity. In addition, the lower electrode 102 may be formed to have a thickness of 10 to 500 nm on the base substrate 101.

Subsequently, as shown in FIG. 23, a sacrificial insulating layer 103 for forming a first fluid reservoir 11a (see FIG. 17) is formed on the lower electrode layer 102. The sacrificial insulating layer 103 is removed through a subsequent process to form the first fluid reservoir 11a in place, and may be formed of, for example, a silicon nitride film (Si3N4). The sacrificial insulating layer 103 may be formed by Plasma Enhanced Chemical Vapor Deposition PECVD or CVD, and may be formed to have a thickness of approximately 100 to 1000 nm in conside the internal volume of the first fluid reservoir to be formed.

Subsequently, a photolithography process is performed to determine the internal volume of the first fluid reservoir 11a, and the sacrificial insulating layer is patterned to have a desired shape.

As shown in FIG. 24, a photoresist film 104 is applied to have a predetermined thickness. The photoresist film 104 may be formed For example, a photoresist for Ultra Violet (UV) or a photoresist for Deep UV (DUP) may be used. Here, the photoresist for UV includes g-line (436 nm) and i-line (356 nm), and the photoresist for DUV includes KrF (248 nm) and ArF (193 nm).

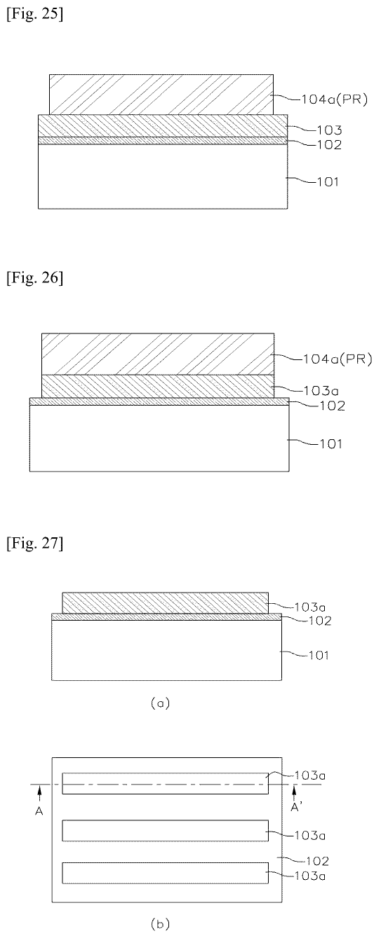

The photoresist film 104 is patterned to a desired shape by exposing the photoresist film 104 using any one of the above-mentioned lithographic light sources. Through this, the photoresist film pattern 104a patterned in the shape of FIG. 25 is formed. Thereafter, as shown in FIG. 26, an etching process using the photoresist film pattern 104a as an etching mask is performed to etch the sacrificial insulating film 103 exposed without being covered by the photoresist film pattern 104a to form a sacrificial insulating layer pattern 103a having a shape corresponding to the shape of the photoresist pattern film 104a.

Subsequently, as shown in FIG. 27, the photoresist film pattern 104a used as the etching mask is removed. FIG. 27(a) is a cross-sectional view of “A-A” shown in (b), and (b) is a plan view.

Subsequently, as shown in FIG. 28, to pattern the lower electrode layer 101, a photoresist film (not shown) is applied on the structure shown in FIG. 27 to cover the upper portion of the sacrificial insulating layer pattern 103a. Thereafter, the photoresist film is patterned using a lithographic mask (not shown) to form a photoresist film pattern 105 in which one side portion (left side portion in FIG. 28) is vertically aligned to one side portion (left side portion in FIG. 28) of the sacrificial insulating layer pattern 103a.

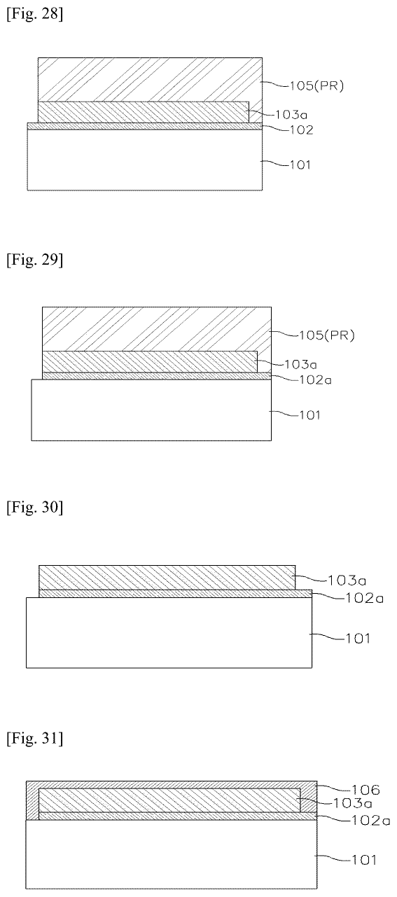

Subsequently, as shown in FIG. 29, the photoresist film pattern 105 is etched by etching the lower electrode layer 102 as the etching mask to form the lower electrode layer pattern 102a. In this case, the etching process for forming the lower electrode layer pattern 102a may be performed by a dry or wet etching process.

Subsequently, as shown in FIG. 30, the photoresist film pattern 105 is removed, for example, a cleaning process is performed to complete the drain electrode (NPHV, see FIG. 18) on the base substrate 101.

Subsequently, as shown in FIG. 31, the lower insulating film 106 functioning as the sidewall and the ceiling of the first fluid reservoir (11a, see FIG. 17) is formed on the base substrate 101 to cover the lower electrode layer pattern 102a functioning as the drain electrode (NPHV). The lower insulating film 106 may be formed by CVD or thermal oxidation, and the deposition thickness may be approximately 10 to 500 nm.

Subsequently, as shown in FIG. 32, the nanopore film 107 is formed on the upper portion of the lower insulating film 106. The nanopore film 107 may be formed as an insulating film based on silicon. For example, the nanopore film 107 may be formed as an insulating film having an etching selectivity with the lower insulating film 106. Preferably, the silicon nitride film (Si3N4) having the same etching selectivity with the sacrificial insulating layer pattern 103a, and may be formed to have a thickness of approximately 5 to 20 nm.

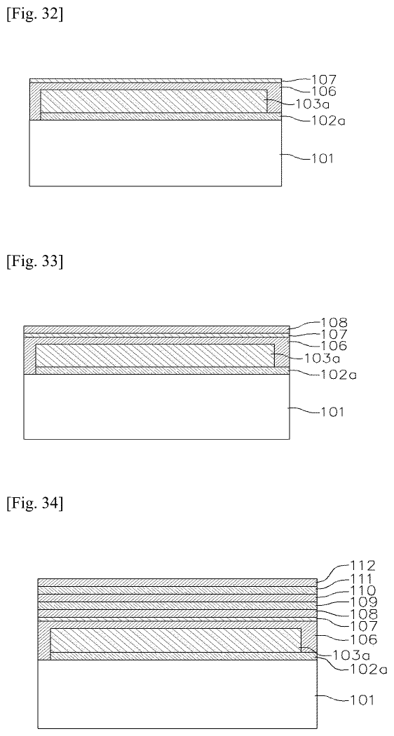

Subsequently, as shown in FIG. 33, the upper insulating film 108 is formed on the upper portion of the nanopore film 107. The upper insulating 108 may be formed to protect the upper portion of the nanopore film 107, and may be formed to have a thickness of approximately 10 to 100 nm.

Subsequently, as shown in FIG. 34, the upper insulating 108 sequentially forms the first upper electrode layer 109, the separation insulating 110, the second upper electrode layer 111 and the protective insulating 112.

The first upper electrode layer 109 is an electrode layer functioning as the control electrode CE1 shown in FIG. 18, and can be formed to a thickness of approximately 10 to 100 nm using the same deposition method and material as the lower electrode layer pattern 102a. The second upper electrode layer 111 is an electrode layer that functions as the source electrode NPGND shown in FIG. 18, and may be formed to a thickness of approximately 10 to 100 nm by using the same deposition method and material as the first upper electrode layer 109.

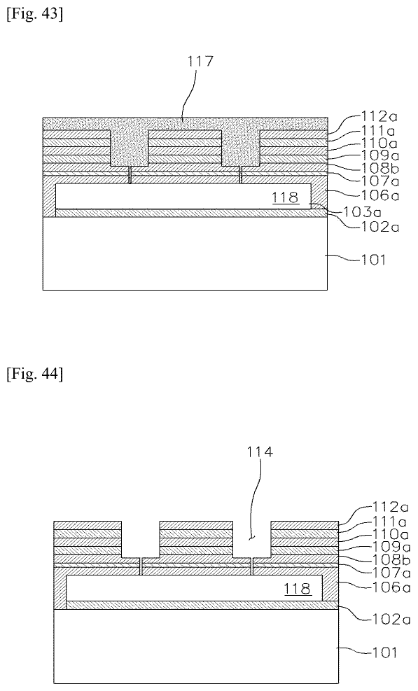

As shown in FIG. 44, the separation insulating film 110 is an insulating film that electrically separates the first and second upper electrode layers 109 and 111, and can be formed to a thickness of approximately 10 to 100 nm using, for example, a buffer oxide film. The protective insulating film 112 is an insulating film that protects the second upper electrode layer 111 and can be formed to a thickness of 10 to 100 nm using a silicon oxide film.

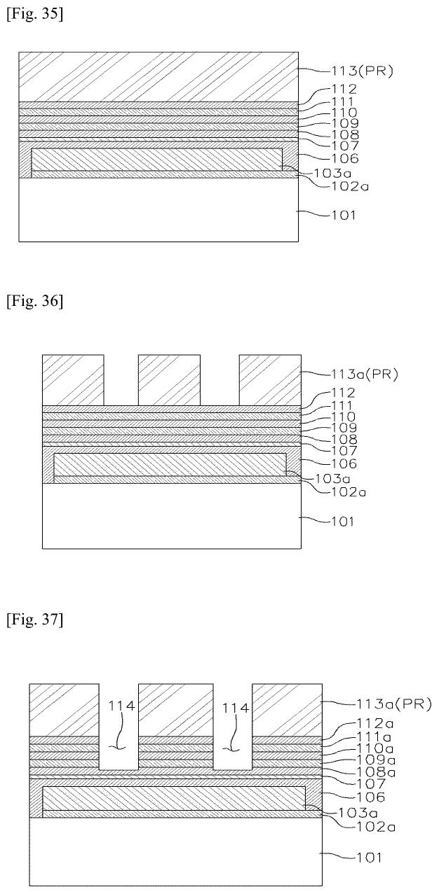

Subsequently, as shown in FIG. 35, after coating photoresist film 113 on the protective insulating film 112, patterning is performed using a lithography mask (not shown) to form photoresist film pattern 113a having the same structure as in FIG. 36. Thereafter, as shown in FIG. 37, a hole 114 is formed by sequentially etching the protective insulating film 112, the second upper electrode layer 111, the separation insulating film 110, and the first upper electrode layer 109 using the photoresist film pattern 113a.

As shown in FIG. 37, when forming the hole 114, a part of the upper portion of the upper insulating film 108 may be etched. Thereafter, when the photoresist film pattern 113a is removed as shown in FIG. 38, the sidewalls are partitioned by the protective insulating film pattern 112a, the second upper electrode layer pattern lila, the separation insulating film pattern 110a, and the first upper electrode layer pattern 109a, and the bottom is formed with the hole 114 partitioned by the upper portion of the upper insulating film pattern 108a. The hole 114 may be formed in the same structure as (b) of FIG. 38 (a stereogram schematically shown to help understand the structure of the hole 114), and as shown in FIG. 18, it may function as a second fluid reservoir 12a, and a conductive electrolyte is filled.

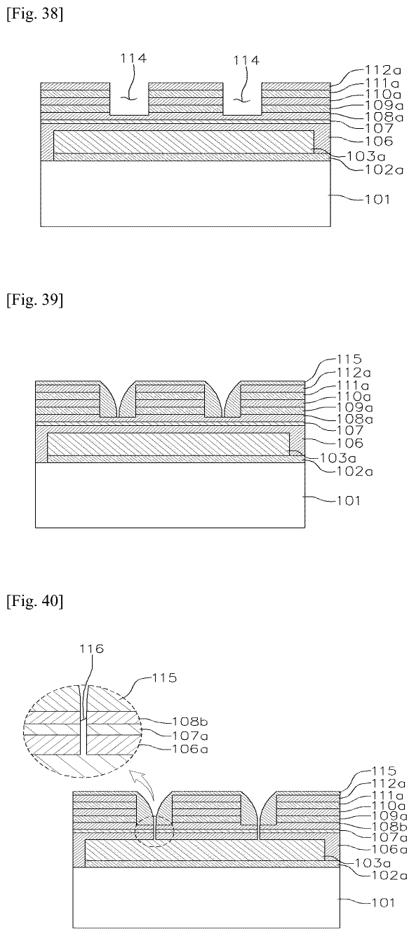

Subsequently, as shown in FIG. 39, a spacer 115 is formed on the inner wall of the hole 114 such as the protective insulating film pattern 112a, the second upper electrode layer pattern 111a, the separation insulating film pattern 110a, and the sidewall of the first upper electrode layer pattern 109a. The spacer 115 functions as a mask during the etching process to form a single nanopore 116 on the nanopore membrane 107. The spacer 115 may form a spacer film to cover the entire structure and then perform an etchback process to form the spacer 115 selectively only on the inner wall of the hole 114. In this case, the spacer 115 may remain partially on the upper portion of the protective insulating film pattern 112a as shown in FIG. 39.

The spacer film for forming the spacer 115 may be appropriately selected in consideration of the etching selectivity with the film to be etched. For example, a metal material or an insulating material may be used. For example, a metal, a metal oxide film, or a metal nitride film may be used as a metal material. Specifically, titanium (Ti), a titanium nitride film (TiN), tungsten (W), and the like can be used, and a silicon nitride film or a silicon oxide film can be used as an insulating material. In addition, various materials may be used depending on the etching selectivity. The deposition thickness of the spacer film can be formed to be appropriate depending on the diameter of the single nanopore 116 to be formed. As the etch-back process, a wet or dry etching process may be used.

Subsequently, as shown in FIG. 40, the spacer 115 is used to etch the upper insulating film pattern 108a, the nanopore film 107, and the lower insulating film 106. Accordingly, a single nanopore 116 is formed in the upper insulating film pattern 108b, the nanopore film pattern 107a, and the lower insulating film pattern 106a.

Subsequently, as shown in FIG. 41, a spacer 115 used as a mask is removed when forming a single nanopore 116. The diameter of a single nanopore 116 can be finely adjusted by controlling the spacing of the spacer 115 as it is determined by the spacing of the spacer 115. In this case, the single nanopore 115 may be formed with a diameter of 1 to 5 nm.

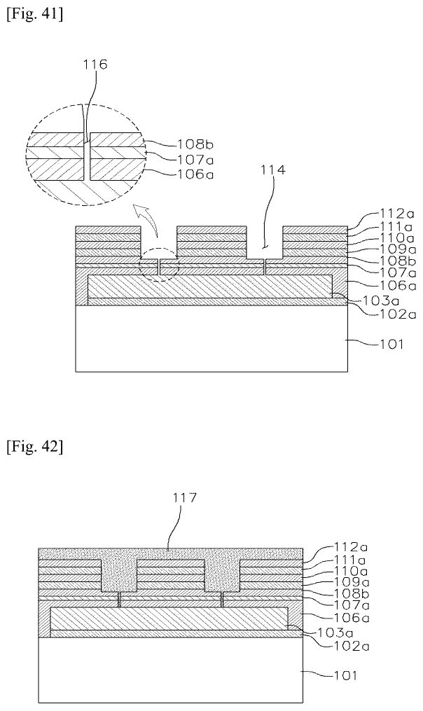

Subsequently, as shown in FIGS. 42 and 43, the sacrificial insulating layer pattern 103a is removed to form a cavity-shaped first fluid reservoir 18. During the removal process of the sacrificial insulating layer pattern 103 a, the diameter of the finely formed single nanopore 116 by the etching solution (echant) should not be affected.

As an example for stably removing the sacrificial insulating layer pattern 103a without affecting the diameter of a single nanopore 116, a sacrificial insulating layer 117 is used. As shown in FIG. 26, the single nanopore 116 and the hole 114 are filled and embedded using the sacrificial insulating film 117 to prevent the diameter of the single nanopore 116 from being expanded or damaged from the etching solution.

As shown in FIG. 43, a single nanopore 116 and a hole 114 are protected by being buried with a sacrificial insulating film 117. In addition, a hole (not shown) in which at least a part of the sacrificial insulating layer pattern 103a is exposed in another portion, for example, the lower insulating layer 106a, is additionally formed, and the sacrificial insulating layer pattern 103a can be removed by etching the sacrificial insulating layer pattern 103a by introducing an etching solution through the holes formed in this manner.

As another example, in the entire structure formed through the process conducted until FIG. 41, the sacrificial insulating film 117 may be deposited so that only a portion of the single nanopore 116 and the hole 114 is embedded, and then the etching solution may be introduced into the sacrificial insulating layer pattern 103 a through a portion where the sacrificial insulating film 117 is not deposited. In this case, a region in which the sacrificial insulating film 117 is not formed may be buried using an insulating film or the like after the removal of the sacrificial insulating layer pattern 103a.

The sacrificial insulating film 117 may be formed of a material having an etch selectivity ratio with the sacrificial insulating layer pattern 103a. For example, it may be made of a metallic material. In the case of forming using a metal material, a spacer is formed on the inner wall of the hole 114 using a silicon oxide film, and the hole 114 can be completely buried using a metal material. It can also be formed of an insulating material instead of a metal material. In the case of forming using an insulating material, a spacer may be formed using a silicon nitride film on the inner wall of the hole 114 and then completely buried with a silicon oxide film.

As shown in FIG. 43, the sacrificial insulating film 117 is masked to remove the sacrificial insulating layer pattern 103a partitioned by the lower electrode layer pattern 102a and the lower insulating layer pattern 106a to form the first fluid reservoir 118. At this time, the etching process of the sacrificial insulating layer pattern 103a may be performed by a wet etching process using an etchant that can remove the silicon nitride film, and the etching process may be performed through a region in which the sacrificial insulating film 117 is not formed.

As shown in FIG. 44, the sacrificial insulating film 117 is removed.

Meanwhile, the hole 114 may function as the second fluid reservoir 12a in FIG. 1. The upper portion of the hole 114 may be covered and sealed using a separate insulating film or member. In this case, a circular, elliptical, or angular hole communicating with the hole 114 may be formed in the member.

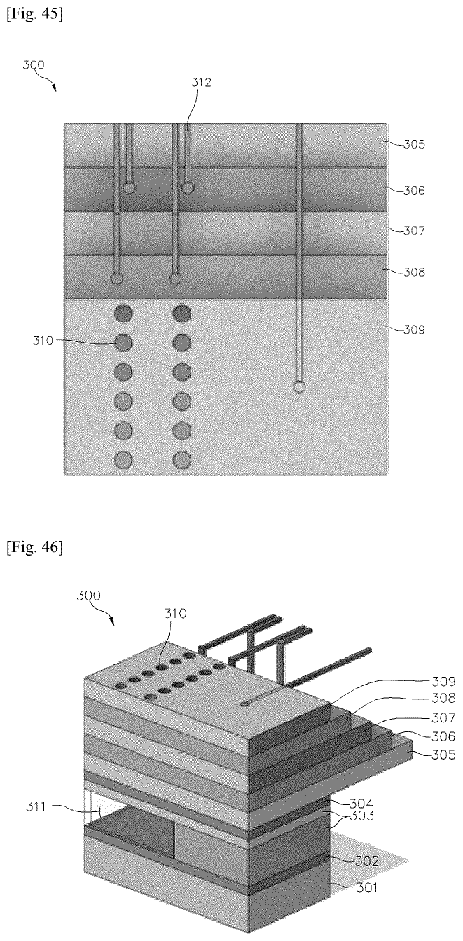

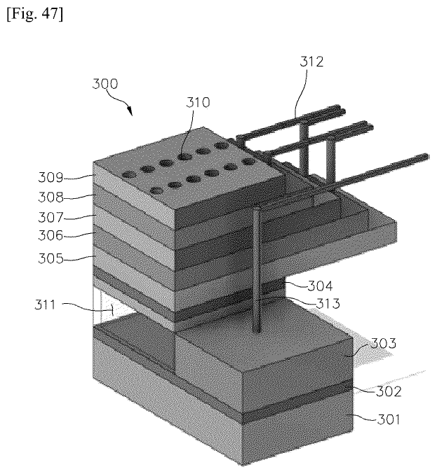

FIG. 45 is a plan view schematically illustrating the DNA chip manufactured through FIGS. 21 to 44, and FIGS. 46 and 47 are cross-sectional perspective views schematically illustrating the DNA chip illustrated in FIG. 45.

As shown in FIGS. 45 to 47, the DNA chip 300 according to the embodiment of the present invention includes a base substrate 301, a drain electrode 302, a lower insulating film 303, a nanopore film 304, an upper insulating film 305, a control electrode 306, a separation insulating film 307, a source electrode 308, a protective insulating film 309, a first fluid reservoir 310, and a second fluid reservoir 311.

The drain electrode 302, the control electrode 306, and the source electrode 308 are respectively connected to the metal line 312. Since the electrodes 302, 306, 308 and the metal line 312 have different positions (heights), the contact plug 313 may be additionally formed to stably connect them. The contact plug 313 may be formed by a method of forming a contact hole or a via hole in a structure and then filling a metal material in the holes.

Although the embodiments of the present specification are described with reference to the accompanying drawings, it will be understood by those skilled in the art to which the present specification pertains that the present invention may be implemented in other specific forms without changing the technical spirit or essential characteristics thereof. Therefore, it should be understood that the embodiments described above are illustrative in all aspects and are not restrictive.

1. A DNA encoding method comprising: (a) converting, by a processor, binary data into sequence information by using a basic base value and a degenerate base value.

2. The method of claim 1, wherein the degenerate base value is a combination of at least two basic base values among four basic bases.

3. The method of claim 1, further comprising: (b) generating, by the processor, a type of DNA strand to be generated in the DNA synthesis step according to the number of basic base values used to express the degenerate base value.

4. The method of claim 3, further comprising: (c) uniformly distributing, by the processor, the total number of DNA strands to be generated in the DNA synthesis step according to the type of DNA strands to be generated in the DNA synthesis step.

5. A DNA encoding apparatus comprising: a processor that converts binary data into sequence information by using a basic base value and a degenerate base value.

6. The DNA encoding apparatus of claim 5, wherein the degenerate base value is a combination of at least two basic base values among four basic bases.

7. The DNA encoding apparatus of claim 5, wherein the processor further generates a type of DNA strand to be generated in the DNA synthesis step according to the number of basic base values used to express the degenerate base value.

8. The DNA encoding apparatus of claim 7, wherein the processor further distributes the total number of DNA strands to be generated in the DNA synthesis step according to the type of DNA strands to be generated in the DNA synthesis step.

9. A DNA decoding method comprising: (a) receiving, by the processor, base sequence analysis information for a plurality of DNA strands; (b) dividing, by the processor, the basic base value and the degenerate base value according to the base type for the same position in the plurality of DNA strands; and (c) converting, by the processor, the basic base value and the degenerate base value into binary data.

10. The method of claim 9, wherein the degenerate base value is a combination of at least two basic base values among four basic bases.

11. The method of claim 9, wherein the step (b) is a step of dividing, by the processor, the basic base value and the degenerate base value according to the base type ratio for the same position in the plurality of DNA strands.

12. The method of claim 11, wherein the step (b) is a step of excluding, by the processor, the base type less than the preset reference number from the basic base value and the degenerate base value.
